(19) United States
(12) Patent Application Publication
(10) Pub. No.: US 2021/0005327 A1
(43) Pub. Date: Jan. 7, 2021

(54) METHOD AND SYSTEM FOR PERSONALIZED, MOLECULAR BASED HEALTH MANAGEMENT AND DIGITAL CONSULTATION AND TREATMENT

(71) Applicant: Molecular You Corporation, Vancouver (CA)

(72) Inventors: Mohammad Ashraful Anwar, Vancouver (CA); Ana Gabriela Marcu, Edmonton (CA); Nitya Bourdel, Vancouver (CA); Robert Allan Fraser, North Vancouver (CA)

(21) Appl. No.: 16/920,514

(22) Filed: Jul. 3, 2020

Related U.S. Application Data
(60) Provisional application No. 62/871,040, filed on Jul. 5, 2019.

(51) Int. Cl. G16H 50/70; C12Q 1/6827; G01N 33/68; G16H 50/30; G16H 10/60; G16H 10/40; G16H 50/20; G16H 70/60; G16H 20/60; G16H 20/10; G16H 20/30; G16H 15/00

(52) U.S. Cl. CPC G16H 50/70; C12Q 1/6827; G01N 33/6803; G16H 50/30; G16H 10/60; G16H 15/00; G16H 50/20; G16H 70/60; G16H 20/60; G16H 20/10; G16H 20/30; G16H 10/40

(57) ABSTRACT

The present disclosure relates to personalized health, specifically molecular based health management and digital consultation. In particular, the present disclosure is directed to methods and systems for assessing the health status of an individual based on correlations between multi-omics measures (e.g., genomics, metabolomics, exposomics and proteomics) and diseases or health risks as disclosed in published research data. The disclosure also relates to methods and systems for customized counseling to individuals regarding health status and actionable measures to improve their health status.

### TECHNICAL FIELD

The present disclosure relates generally to systems and methods for digital medical profiling and/or evaluating health status, and patient consultation. In particular, the disclosure is directed to personalized molecular health profiling, diagnosis, monitoring and/or remedy prescription and methods of treatment thereof.

### BACKGROUND

The field of personalized health (also known as personalized medicine or precision health precision medicine) has been gaining attention, particularly with respect to: (i) preventative medicine and early detection and treatment of diseases; and (ii) optimization of health, fitness and nutrition. Personalized health involves measurements of multiple biological parameters, which in combination with bioinformatics allows healthcare professionals and/or individuals to accurately assess an individual's current health status, disease risk, fitness and/or how to best mitigate the risks. In fact, understanding an individual's overall health status plays an important role in patient counseling with actionable recommendations to help reduce, ameliorate and/or prevent disease risks and/or optimize health/performance customized for that individual.

Recent advances in high-throughput bioscience technologies have led to the possibility of a more precise modeling of disease risk for a given individual and situation. For instance, biomarkers play a key role in diagnosing, profiling and/or managing these disease risks. There is a plethora of published research information available on biomarkers and their associated disease risks. However, there are challenges correlating the information to the health status and/or disease risks. Additionally, some of the data may be contradictory to one another. Further, the data may be isolated from other relevant health information, such that it does not provide an objective measure of an individual's overall health status.

Moreover, new research information is constantly being published and updated on an annual, if not, monthly basis by different research groups around the world. Therefore, it is important that a method exists to consistently, accurately and dynamically evaluate the strength of the research evidence between the biomarkers that are linked to disease risks in order to be able to derive reliable and useful information therefrom. Once in possession of such information, the patient can then directly, or indirectly, with the help of health professionals (e.g., physicians, clinicians, dieticians, therapists, etc.), make an informed decision of the type of actionable measures (including changes in medications and nutritional supplements, and lifestyle interventions such as diet and exercise), that could be useful to maximize his/her health status or as a preventive measure to delay the progress of diseases.

Assessing and evaluating the performance value of published research information, particularly newly published research papers, specifically, in terms of their reproducibility of results, has remained a critical, increasingly necessary and important issue with no acceptable existing solution. Currently, a variety of metrics are employed, such as for example: (i) citation score which is primarily used for research papers; (ii) impact factor (IF) (also known as journal impact factor (JIF)) which is mainly used for journals; and (iii) scientific H-index (also known as H-factor or H-value) which is mainly used for researchers. Almost all of these metrics are based on a determination of the citation received (i.e., cited by what publication and/or researcher and the number of citations), which are then presumed to correlate to the reproducibility of the published research results.

As noted above, one approach has been to use a citation score. The citation score reflects the number of citations of the first research paper by the second paper and optionally the influence of the second paper is taken into account in the citation score. Another approach has been to rely on impact factor, which measures the yearly average number of citations to recent articles published in that journal and serves as a proxy for the relative importance of a journal within its field. A yet further approach has been to rely on scientific reputation based on the generally known H-index, which is an index that attempts to measure both the productivity and impact of the published work of a scientist or scholar. For example, a researcher with a large H-index may have a significant amount of prestige and influence within the research community.

These metrics have limited value, however, because they have a host of common issues that call into question their effectiveness. Firstly, the metrics are not readily comparable across different fields of science or even different types of papers. For example, it is believed that published review articles rather than scientific research papers, clinical papers or papers directed to single case studies will be more helpful to increase the number of citations, impact factor and/or even H-index of a publication. Secondly, researchers may be biased and tend to work in “hot” disciplines or trending research areas that may potentially lead to more publications or attract more citations. Lastly, some researchers tend to cite articles or publications only from particular collaborators or organizations, which typically often include the authors themselves. Such practice is commonly referred to as “self-citation”, and is used to further enhance a researcher's metric scores. As a result, these metrics and the methods that employ such metrics fail to accurately correlate the biomarkers to the associated disease risks.

An improved method of assessing health status, preferably overall health status, which provides meaningful and accurate information to aid in patient consultation, is needed. A need also exists for a system for assessing the health status for predicting a subject's risk of developing certain diseases in the future based on current information.

### SUMMARY

As embodied described herein, in one aspect, the present disclosure relates to a method for assessing the health status of a human subject. The method comprises: providing a biological sample obtained from the individual; measuring at least 25, preferably at least 20, preferably at least 15, preferably at least 10 or preferably at least 5 Disease Risk Markers in the biological sample selected from the group consisting of Genomic Markers, Proteomic Markers, Metabolomic Markers, Exposomic Markers and a combination thereof to provide measurement data from the sample in relation to the individual; and determining a predicted health status corresponding to a disease or health risk or a risk of developing thereof, by applying a predictive equation corresponding to the disease or health risk or the risk of developing thereof to the measurement data. The predictive equation corresponds to the disease or health risk or the risk of developing thereof and is determined by multivariate regression analysis of published data of human subjects that have the disease or the health risk. The multivariate regression analysis comprises calculating a confidence score of each of the published data of the human subjects and the published data comprises a plurality of measurements corresponding to each human subject that have the disease or health risk. The plurality of measurements correspond to each Disease Risk Marker associated with the disease or health risk and determined from published Disease Risk Markers of each human subject in the published data. The predicted health status is representative of the individual having the disease or health risk or the risk of developing thereof.

As embodied and described herein, in another aspect, the present disclosure also relates to a method of determining a health status of an individual, based on a set of Disease Risk Markers corresponding to a disease or a health risk and a magnitude of a gap between measured Disease Risk Markers and published Disease Risk Markers. The method comprises: analyzing at least 25, preferably at least 20, preferably at least 15, preferably at least 10 or preferably at least 5 sampled Disease Risk Markers of the individual to determine measurement data indicative of a disease or health risk or a risk of developing thereof of a human subject, wherein the at least 25, preferably at least 20, preferably at least 15, preferably at least 10 or preferably at least 5 measurement data corresponds to the disease or health risk; determining the absence or presence of polymorphisms in the sampled Disease Risk Markers or levels of the sampled Disease Risk Markers from the measurement data from the individual; and calculating, by a computer device, and based on the at least 25, preferably at least 20, preferably at least 15, preferably at least 10 or preferably at least 5 measurement data, a magnitude of a gap between the sample Disease Risk Markers and corresponding published Disease Risk Markers. Each Disease Risk Marker is correlated with affecting one or more of the disease or health risk and the magnitude of the gap indicates the health status of the individual.

As embodied and described herein, in yet another aspect, the present disclosure also relates to a method for assessing Body Functions of an individual. The method comprises: providing a biological sample obtained from the individual; measuring at least 25, preferably at least 20, preferably at least 15, preferably at least 10 or preferably at least 5 Disease Risk Markers in the biological sample selected from the group consisting of Genomic Markers, Proteomic Markers, Metabolomic Markers, Exposomic Markers and a combination thereof to provide measurement data from the sample in relation to the individual; and determining a predicted health status corresponding to the Body Functions, by applying a predictive equation corresponding to the measurement data to the Body Functions. The predictive equation corresponds to the Body Functions and is determined by multivariate regression analysis of published data of human subjects that have the disease or health risk. The multivariate regression analysis comprises calculating a confidence score of each of the published data of the human subjects and the published data comprises a plurality of measurements corresponding to each human subject to the Body Functions. The measurements are associated with biological pathways involving a complex network of Genomic Markers, Proteomic Markers, Metabolomic Markers, and/or Exposomic Markers, and determined from published Disease Risk Markers of each human subject in the published data. The predicted health status is representative of the Body Functions of the individual.

As embodied and described herein, in yet another aspect, the present disclosure also relates to a method of assessing the health status of an individual. The method comprises: providing a biological sample obtained from the individual, measuring at least 25, preferably at least 20, preferably at least 15, preferably at least 10 or preferably at least 5 Disease Risk Markers in the biological sample selected from the group consisting of Genomic Markers, Proteomic Markers, Metabolomic Markers, Exposomic Markers and a combination thereof to provide measurement data from the sample in relation to the individual, and determining a predicted health status corresponding to a disease or health risk or a risk of developing thereof, by applying a predictive equation corresponding to the disease or health risk or the risk of developing thereof to the measurement data. The predictive equation is determined by multivariate regression analysis of published data of human subjects that have the disease or health risk. The multivariate regression analysis comprises calculating a first confidence score of each of the published data of the human subjects, wherein the first confidence score relates to a measure of confidence on the strength of predictiveness of the published data used to determine the likelihood of having or at risk of developing the disease or health risk. The published data comprises a plurality of measurements corresponding to each human subject that have the disease or health risk. The measurements correspond to each Disease Risk Marker associated with the disease or health risk and determined from published Disease Risk Markers of each human subject in the published data. The predicted health status is representative of the individual having the disease or health risk or the risk of developing thereof.

As embodied and described herein, in yet another aspect, the present disclosure also relates to a system for performing any one of the methods as described herein.

As embodied and described herein, in yet another aspect, the present disclosure also relates to a system (100) for assessing the health status of an individual. The system (100) comprising: at least one processor (104); an interface (106); and at least one tangible, non-transitory computer readable storage medium storing computer executable instructions (108). The instructions (108) when executed by the at least one processor (104), cause the system (100) to: obtain, via a Disease Risk Markers measurement provider (115), an indication of the presence, absence or level of Disease Risk Markers in a biological sample from the individual, wherein the Disease Risk Marker is selected from the group consisting of Genomic Markers, Proteomic Markers, Metabolomic Markers, Exposomic Markers and a combination thereof; and determine, based on the indication of the presence, absence or level of the sampled Disease Risk Markers, a predicated health status corresponding to a disease or health risk or a risk of developing thereof, by applying a predictive equation corresponding to the sampled Disease Risk Markers. The predictive equation is determined by multivariate regression analysis of published data of human subjects that have the disease or health risk. The multivariate regression analysis comprises calculating a first confidence score of each of the published data of the human subjects, wherein the first confidence score relates to a measure of confidence on the strength of predictiveness of the published data used to determine the likelihood of having or at risk of developing the disease or health risk, and the published data comprises a plurality of measurements corresponding to each human subject that have the disease or health risk. The measurements are associated with the disease or health risk and determined from published Disease Risk Markers of each human subject in the published data. The health status is representative of the individual having the disease or health risk or risk of developing thereof.

As embodied and described herein, in yet another aspect, the present disclosure also relates to a system (120). The system (120) comprises: a) a database (121) comprising published data of Disease Risk Markets associated with a disease or health risk in human subjects, wherein the Disease Risk Markers are selected from group consisting of: Genomic Markers, Proteomic Markers, Metabolomic Markers, Exposomic Markers and a combination thereof; and b) a computer (122) comprising computer readable instructions for determining a first confidence score of each of the published data, wherein the first confidence score indicates a likelihood of an association of the Disease Risk Markers to the disease or health risk in the published data is reproducible. The computer readable instructions: (i) generate relational data to represent a relationship between each of the published Disease Risk Marker and the association; and (ii) uses the relational data to determine the confidence score for the association.

As embodied and described herein, in yet a further aspect, the present disclosure relates to a method for assessing the health status of, the method comprising: (i) providing a biological sample obtained from the; (ii) measuring at least 25, preferably at least 20, preferably at least 15, preferably at least 10 or preferably at least 5 Disease Risk Markers in the biological sample selected from the group consisting of Genomic Markers, Proteomic Markers, Metabolomic Markers, Exposomic Markers and a combination thereof to provide collected measurement data from the sample in relation to the; (iii) inputting the collected measurement data to a computer-implemented data processing system; (iv) processing the collected measurement data in the data processing system by assigning individual biomarker levels to respective entries in a plurality of electronic data entries in a database corresponding to published data of Disease Risk Markers associated with a disease or health risk in human subjects, wherein the Disease Risk Markers are selected from group consisting of: Genomic Markers, Proteomic Markers, Metabolomic Markers, Exposomic Markers and a combination thereof; (v) outputting a predicted health status corresponding to a disease or health risk or a risk of developing the disease or risk thereof, by applying a predictive equation corresponding to the disease or health risk or the risk of developing thereof to the collected measurement data, the predictive equation having been determined by a computer-implemented multivariate regression analysis of published data of human subjects that have the disease or health risk, the multivariate regression analysis comprising outputting a confidence score of each of the published data of the human subjects, wherein the published data comprises a plurality of measurements corresponding to each human subject that has the disease or health risk, the plurality of measurements correspond to each Disease Risk Marker associated with the disease or health risk and are determined from published Disease Risk Markers of each human subject in the published data, the predicted health status being representative of the individual having the disease or health risk or the risk of developing thereof; and (vi) displaying the predicted health status on an electronic display connected directly or wirelessly to the data processing system.

In one embodiment, the measuring step (ii) comprises at least one step of mass spectrometry. In another embodiment, the collected measurement data is input to a database. According to a further embodiment, the confidence score is based on an output from a return-on-bibliography (ROB) score. In a further embodiment, the method further comprises determining disease risk scores based on a magnitude of the gap technique. In yet a further embodiment, the confidence score is a weighted score computed by stacking an initial confidence score with one or more additional confidence scores.

In one aspect, the Applicant has found that a combination of multiple reaction monitoring mass spectrometry, high performance liquid chromatography, and liquid chromatography-mass spectrometry can achieve the most accurate, quantifiable, and reliably consistent biomarker levels results. Thus, the present disclosure relates to any one of the above-described aspects and/or embodiments of the disclosure in which biomarkers are measured using one or a combination of mass spectrometry, high performance liquid chromatography, and liquid chromatography-mass spectrometry. In one embodiment, the analysis comprises at least one step of mass spectrometry, which may be carried out in a mass-spectrometry unit, optionally coupled with another analytical technique.

In yet another aspect, the present disclosure relates to a method of treating a disease or condition in a subject, comprising: determining a health status of an individual based on any of the method disclosed herein, wherein said health status is indicative of the progression of the disease or condition, and recommending changes in medication, supplements and/or nutrition for the individual to treat the disease or condition. In an embodiment, the disease or condition is selected from the group consisting of psoriasis, crohn's disease, bipolar disorder, depression, schizophrenia, age-related macular degeneration, adolescent idiopathic scoliosis, hurler syndrome, tooth agenesis, celiac disease, multiple sclerosis, vas deferens condition, asthma, allergic rhinitis, heroin addition, low bone mineral density, osteoporosis, gout, ADHD, ulcerative colitis, pancolitis, post-traumatic stress disorder, autism, type 1 diabetes, type 2 diabetes, renal cell carcinoma, peanut allergy, Fuch's dystrophy, Creutzfeldt-Jakob disease, hepatitis C, obsessive-compulsive disorder, coronary artery disease, cardiovascular disease, pancreatic cancer, systemic lupus erythematosus, rheumatoid arthritis, cocaine dependence, deep vein thrombosis, Hirschsprung disease, nicotine dependence, diabetic nephropathy, ischemic stroke, T2D, autoimmune disease, several alcohol withdrawal, Atrial Fibrillation, ankylosing spondylitis, melanoma, ALS, migraine-associated vertigo, endometrial ovarian cancer, coronary heart disease, Parkinson's Disease, lung cancer, prostate cancer, childhood-onset steroid-sensitive nephrotic syndrome, schizophrenia, phobic disorders, Graves' disease, obesity, wet ARMD, docetaxel-induced nephropathy, pulmonary tuberculosis, male pattern baldness, bipolar disorder, CRP, osteoarthritis, Parkinson's Disease, serum uric acid concentration, myocardial infarction risk, intracranial aneurysm risk, metabolic syndrome, spondylitis, hyper triglyceride, lupus, ischemic stroke, otosclerosis, cutaneous melanoma, ADHA, non-alcoholic fatty liver disease, atherosclerotic cerebral infarction, restless legs syndrome, narcolepsy, temporomandibular joint disorder (TMD), colorectal cancer, Ankylosing Spondylitis, neuroticism, panic disorder, venous thrombosis, glaucoma, hereditary hemochromatosis, Bechet's disease, hypertension, insulin sensitivity, anorexia, Tourette's syndrome, primary biliary cirrhosis, intracranial aneurysm, vitiligo, alcohol dependence, glioma, high blood pressure, hyperuricemia, pulmonary tuberculosis, spondylitis, venous thromboembolism, lumbar disc disease, cardiomyopathy, primary sclerosing cholangitis, colorectal caner, esophageal cancer and breast cancer.

All features of exemplary embodiments which are described in this disclosure and are not mutually exclusive can be combined with one another. Elements of one embodiment can be utilized in the other embodiments without further mention. Other aspects and features of the present disclosure will become apparent to those ordinarily skilled in the art upon review of the following description of specific embodiments in conjunction with the accompanying Figures.

### BRIEF DESCRIPTION OF THE DRAWINGS

While the specification concludes with claims particularly pointing out and distinctly claiming the disclosure, it is believed that the disclosure will be better understood from the following description of the accompanying figures wherein:

FIG. 1 is a flow diagram of a method (10) of assessing the health status of an individual according to an illustrative embodiment of the present disclosure.

FIG. 2 is a schematic illustration of a system according to an illustrative embodiment of the present disclosure.

FIG. 3 is a visualization of the body function assessment with the Disease Risk Markers according to an embodiment of the present disclosure.

FIG. 4 is a Sankey diagram visualizing the links between lifestyle action plan (i.e., health recommendation) with the Disease Risk Markers.

FIG. 5 is a graph displaying an exemplary distribution of ROB scores generated for published research papers according to one aspect of the present disclosure. Many research papers have low ROB scores while only a few have high ROB scores. The distribution is segmented into 4 quartiles that were used to assign confidence scores (or confidence intervals) corresponding to each Disease Risk Marker to disease association.

FIG. 6 is flowchart that represents the overall process of how a risk score is calculated for each Disease Risk Marker. These Disease Risk Marker risk scores are aggregated together to form health risks and lifestyle action plan recommendations that are auto-generated into a final health report that is reviewed by scientists before finally being shared with the client.

FIG. 7 is an exemplary study design of a proof-of-concept study where three cohorts of 50 participants each (total 150 study participants) were given health reports and lifestyle action plans to determine if the action plans can positively impact health over time.

FIG. 8 are charts displaying aggregate information of these study participants that show around 20% of the cohort displayed moderate and high health risks for various diseases, including type 2 diabetes and Alzheimer's disease. The line graph displays the aggregate health risk results for these participants at the start of the study and after 100 days of following the action plan, which shows complete reduction of health risks in the various diseases.

FIG. 9 are charts displaying aggregate information of these study participants that show that the majority of study participants (68%) have abnormal levels of Disease Risk Markers that are typically associated as early indicators and/or casual factors for many chronic diseases. The line graph displays the aggregate body functions risk (also referred to as organ health) status results for these participants at the start of the study and after 100 days of following the action plan, which shows complete reduction of body functions risks that are associated with abnormal Disease Risk Marker levels for early indicators and/or causal factors of disease.

FIG. 10 depicts a schematic for various levels of confidence in association of the Disease Risk Markers to the disease or health risk in the published data and/or controlled experiments and the impact of the Disease Risk Markers to the health recommendation system.

In the drawings, exemplary embodiments are illustrated by way of example. It is to be expressly understood that the description and drawings are only for the purpose of illustrating certain embodiments and are an aid for understanding. They are not intended to be construed as limiting to the invention in any manner.

### DETAILED DESCRIPTION

A detailed description of one or more embodiments of the invention is provided below along with accompanying figures that illustrate the principles of the invention. The invention is described in connection with such embodiments, but the invention is not limited to any particular embodiment described herein. The scope of the invention is limited only by the claims. Numerous specific details are set forth in the following description in order to provide a thorough understanding of the invention. These details are provided for the purpose of providing non-limiting examples and the invention may be practiced according to the claims without some or all of these specific details. For the purpose of clarity, certain technical material that is known in the technical fields related to the invention has not been described in detail so that the invention is not unnecessarily obscured by such descriptions.

### Definitions

Unless otherwise defined, all technical and scientific terms used herein have the same meaning as commonly understood by a person of ordinary skill in the art to which the present invention pertains. As used herein, and unless stated otherwise or required otherwise by context, each of the following terms shall have the definition set forth below.

Articles such as “a” and “an” when used in a claim, are understood to mean one or more of what is claimed or described.

The term “biomarker” or “marker” are used interchangeably herein to mean a substance that is used as an indicator of a biological state (e.g., genes, mRNA, microRNAs (miRNAs), proteins, metabolites, sugars, fats, metals, minerals, nutrients, toxins, etc.).

The terms “comprises”, “comprising”, “include”, “includes”, “including”, “contain”, “contains” and “containing” are meant to be non-limiting, i.e., other steps and other sections which do not affect the end of result can be added. The above terms encompass the terms “consisting of” and “consisting essentially of”.

The term “disease” generally refers to a disorder or particular abnormal condition that negatively affects the structure or function in an organism (e.g., human), especially one that produces specific signs or symptoms often construed as medical conditions. Disease may be caused by external factors (e.g., pathogens) or by internal dysfunctions. Non-limiting examples of diseases include cancer, diabetes, heart disease, allergies, immunodeficiency and asthma.

The term “Disease Risk Markers” generally refer to multi-omics measures (e.g., genomic, proteomic, metabolomics and exposomic) associated with having or developing a disease or health risk in an organism (e.g., human). Disease Risk Markers may also be used to characterized Body Functions in an organism.

The term “Exposomic Markers” generally refer to biomarkers that provide information indicative of environmental exposures experienced by an individual including climate, lifestyle factors (e.g., tobacco, alcohol), diet, physical activity, contaminants, radiation, infections, etc. Exposomic Markers may also provide information indicative of an individual's environment, such as the location of the individual's residence, the quality of the residence, etc. that may have an impact on the individual's health. It will be understood that Exposomic Markers are dynamic and their results are affected for example by changes in the environmental factors. Suitable examples of “Exposomic Markers” are described in the specification herein below.

The term “Genomic Risk Markers” generally refer to one or a set of signature genetic variants on the DNA of an individual and direct inference of causality of a disease or health risk. The types of genetic variants may include insertions or deletions of the DNA at particular locations and single nucleotide polymorphisms (SNPs) in which a particular nucleotide is changed. Genomic Risk Markers are typically considered static (e.g., inherited traits) and do not change over time. However, it is possible in certain instances for Genomic Risk Markers to be dynamic and mutable for example in tumour formation. Evaluation of Genomic Risk Markers obtained from an individual is expected to provide information as to how each variant affects disease pathogenesis and susceptibility to those diseases. Suitable examples of “Genomic Risk Markers” are described in the specification herein below.

The term “health risk” generally refers to an adverse event or negative health consequence due to a specific disease or condition. For example, the health risks of obesity may include diabetes, joint disease, increased likelihood of certain cancers, and cardiovascular disease. All of these are consequences related to obesity and are therefore considered health risks associated with obesity. Health risk may also be related to genetic conditions, chronic diseases, certain occupations (e.g., miners are exposed heavy metal pollutants) or sports (e.g., concussions in football players are linked to memory loss, depression, anxiety, etc.), lifestyle factors (e.g., alcoholics are at higher risk of developing fatty liver) or any number of events or situations

The term “health status” generally refers to a qualitative or quantitative indication of the profile of a respective health status of an individual at the time of evaluation.

The term “Metabolic Markers” generally refer to metabolites and/or metabolite profiles that provide information of metabolic pathways associated with biological conditions and functions in a system in an individual. “Metabolic pathway” refers to a sequence of enzyme-mediated reactions that transform one compound to another and provide intermediates and energy for cellular functions. The metabolic pathway can be linear or cyclic. The functional impact of metabolites and/or metabolite profiles is useful to infer causality of disease or health risks. As a result, Metabolic Markers are useful to accurately identify individual's health status, particularly with reference to a disease or susceptibility to the disease. Metabolic Markers are dynamic and their results are affected for example by changes in health, medication and nutrition. Suitable examples of “Metabolic Markers” are described in the specification herein below.

The term “predicted health status” generally refers to such a quantitative indication of the profile of a respective health status at a later time after the evaluation. For example, when a predicated health status is obtained via DNA analysis, the predicted health status is calculated by applying a predictive equation to the measured Genomic Markers.

The terms “preferred”, “preferably” and variants generally refer to embodiments of the disclosure that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

The term “preventing” or “prevention” generally refers to a reduction in risk of acquiring a disease or health condition. As a result, at least one of the symptoms of the disease or health condition does not develop in an individual that may be exposed or predisposed to the disease or health condition but does not yet experience or display symptoms of the disease or health condition.

The term “Proteomic Markers” generally refer to functional proteins and/or protein profiles that provide information of ongoing physiological, developmental or pathological events in an individual, and that correlate to disease or health risks. While genomic technologies have identified genes specifically related to diseases, the function of such genes and the data interpretation in the context of functional regulation by various process (e.g., proteolytic degradation, posttranslational modification, involvement in complex structures, and compartmentalization) of those genes is aided by the evaluation of Proteomic Markers. “Proteomic Markers” are concerned with looking at a protein repertoire of a defined entity, be it a biological fluid, an organelle, a cell, a tissue, an organ, a system or the whole individual. Evaluation of Proteomic Markers obtained from an individual is expected to increase the understanding and monitoring of disease pathogenesis and susceptibility to those diseases. Proteomic Markers are dynamic and their results are affected for example by changes in health, medication and nutrition. Suitable examples of “Proteomic Markers” are described in the specification herein below.

In all embodiments of the present disclosure, all percentages, parts and ratios are based upon the total weight of the compositions of the present disclosure, unless otherwise specified. All such weights as they pertain to listed ingredients are based on the active level and, therefore do not include solvents or by-products that may be included in commercially available materials, unless otherwise specified.

All ratios are weight ratios unless specifically stated otherwise. All temperatures are in Celsius degrees (° C.), unless specifically stated otherwise. All dimensions and values disclosed herein (e.g., quantities, percentages, portions, and proportions) are not to be understood as being strictly limited to the exact numerical values recited. Instead, unless otherwise specified, each such dimension or value is intended to mean both the recited value and a functionally equivalent range surrounding that value. For example, a dimension disclosed as “40 mm” is intended to mean “about 40 mm.”

### Method of Assessing Health Status

In one aspect, the present disclosure is predicated, at least in part, on the recent advances in high-throughput bioscience technologies that have led to the discovery of correlations between multi-omic measures (e.g., genomics, metabolomics, exposomics and proteomics) and diseases or health risks. In particular, the inventors discovered that evaluation of multi-omic measures of biological parameters to acquire associations with diseases or health risks allows for more accurate assessment of an individual's health status in relation to the diseases or health risks, or prediction of the individual's susceptibility of developing the diseases or health risks.

The complex aetiologies associated with diseases or health risks are influenced by a combination of genetic and environmental factors unique to each individual and condition. Indeed, diseases or health risks are caused by any number of physiological, behavioral and environmental dynamics. Given the broad spectrum of underlying factors that contribute to the causation of diseases or health risks, the identification of multi-omic measures predictive for diseases or health risks was unpredictable. The discovery of a method and system to evaluate published research information to confirm strong correlations between certain multi-omics measures and multiple diseases or health risks allowed accurate assessment of an individual's health status in a manner which has not been achieved previously. Furthermore, the disclosure provides a computer-implemented method and system for providing, customized, “concierge” counseling to individuals about their specific health status. Therefore, the present disclosed subject matter represents an advancement in the art.

As set forth herein, the inventors have discovered surprising correlations between multi-omic measures and diseases or health risks for overcoming the disadvantages as described above. In particular, the inventors have developed a computer-generated scoring metric called return-on-bibliography (ROB) score that can consistently, accurately and dynamically evaluate published research information as to the reproducibility of their published results. Indeed, the ROB score was observed to evolve over time as the research information is updated with newly published research information or as previous research information may be retracted.

Thus, it is an advantage of the present disclosure to provide a new method to objectively evaluate published research information in terms of the reproducibility of the published results. The method is simple to calculate but consistent in its ability to compare across different disciplines (i.e., research fields, including sub-fields) and different types of publications. It is a further advantage of the present disclosure to utilize research information pertaining to multiple types of biomarkers to provide more accurate and complete insights into the individual's overall health status. It is yet a further advantage to increase the individual's acceptance of the results and increase the likelihood of initiating and adhering to lifestyle interventions to mitigate against diseases or health risks. The incorporation of genomic and metabolomics information in a health assessment methodology described herein can have this desirable effect.

Specifically, in one aspect, the present disclosure provides for a method of assessing the health status of an individual. The method comprises measuring at least 25, preferably at least 20, preferably at least 15, preferably at least 10 or preferably at least 5 Disease Risk Markers in the biological sample to provide measurement data from the sample; and determining a predicted health status corresponding to a disease or health risk, or a risk of developing thereof. In certain embodiments, the method comprises measuring at least 300, 275, 250, 225, 200, 175, 150, 125, 100, 95, 90, 85, 80, 75, 70, 65, 60, 55, 50, 45, 40, 35, 30, 35, 20, 15, 10, or 5 Disease Risk Markers in the biological sample.

The Disease Risk Markers are selected from the group consisting of Genomic Markers, Proteomic Markers, Metabolomic Markers, Exposomic Markers and a combination thereof. The predicted health status is determined by applying a predictive equation corresponding to the disease or health risk or the risk of developing thereof to the measurement data. The predictive equation is determined by a multivariate regression analysis of published data of human subjects that have the disease or health risk to calculate a confidence score of each of the published data from the human subjects. The published data comprises a plurality of measurements corresponding to each human subject that have the disease or health risk. The measurements are associated with the disease or health risk and determined from published Disease Risk Markers of each human subject in the published data. In various embodiments, the predicted health status is representative of the individual having the disease or health risk or risk of developing thereof.

Optionally, the method described herein comprises determining a respective predicted health status by measuring at least two, at least three or all four Disease Risk Markers selected from the group consisting of Genomic Markers, Proteomic Markers, Metabolomic Markers and Exposomic Markers. Thus, in some embodiments, the published data of Disease Risk Markers is applied in at least two, at least three or all four different predictive equations to calculate predicted health status that incorporates at least two, at least three or all four of Genomic Markers, Proteomic Markers, Metabolomic Markers and Exposomic Markers. Thus, in one aspect, the method of the disclosure provides information regarding an individual's health status or risk of developing a disease or health risk based on four different biologic biomarkers, which allows a more comprehensive and accurate evaluation of an individual's health status.

In one embodiment, the disclosure provides a method wherein the step of determining the predicted health status further comprises: comparing the measured Disease Risk Markers to the published Disease Risk Markers associated with the disease or the health risk; and determining a magnitude of a gap between the measured Disease Risk Markers and the published Disease Risk Markers. In this regard, it is understood that the larger the magnitude of the gap, the “worse off” the individual's health status is relative to a control group (i.e., human subjects that do not have the disease or health risk). For these individuals, it is advisable that they become aware of their health status in order to ensure actionable measures are recommended/chosen to help reduce or minimize the magnitude of the gap. It is desirable that this information is obtained earlier in the individual's life (e.g., 40 years or below, 35 years or below, 30 years or below, or 25 years or below), so as to increase any benefits from the delay or offset of the progress of the diseases or health risks.

In another embodiment, while a smaller magnitude of gap reflects the individual's better health status up to that point in time, there is no assurance that the magnitude of the gap will continue to remain small at a later time point. This is due in part, for example, to changing physiology of the body, changing medications, changing nutrition and/or lifestyle choices of the individual over time. Therefore, it is advisable for the individual to continually monitor his/her health status on a regular basis. As a result, the method according to the present disclosure also allows the individual to monitor changes in health status over time.

Accordingly, in certain embodiments, the method further comprises determining a respective predicted health status for each of the disease or health risk. Each respective predicted health status is calculated by applying a respective predictive equation to the respective measurement data for each of the respective Disease Risk Markers. In one embodiment, a unique predictive equation for each of the Genomic Markers, Proteomic Markers, Metabolomic Markers and Exposomic Markers, as appropriate, is applied, resulting in, for example, four predictive health status, each of which corresponds to each of the disease or health risk. In one aspect, the predictive equations are based on the respective strengths of correlation of the Disease Risk Markers to the respective disease or health risk.

The method of the present disclosure also preferably further comprises: determining, based on the sampled measurement data of the individual, a respective current health status corresponding to each of the disease or health risk; and determining a respective magnitude of a respective gap between the respective predicted health status and the respective current health status for each of the disease or health risk. If desired, the disease or health risk associated with the largest respective gap magnitude is identified. For example, the method allows identifying a respective current health status indicating a greater severity in the disease or health risk (i.e., worst condition) than would be predicted by the respective predicted health status, and prioritizing the disease or health risk with the largest respective gap magnitude to, e.g., help to select or recommend changes in medications and nutritional supplements, and lifestyle interventions such as diet and exercise.

The method described herein preferably further comprises: determining a subsequent health status of the individual from analysis of a subsequent measurement data of the individual at a later time point; and determining a subsequent magnitude of a gap between the predicted health status and the subsequent health status of the individual. Accordingly, the present disclosed method might also would benefit those individuals whose magnitude of the gap is small, as it is likely that they would want to routinely monitor such gap to ensure that it remains low.

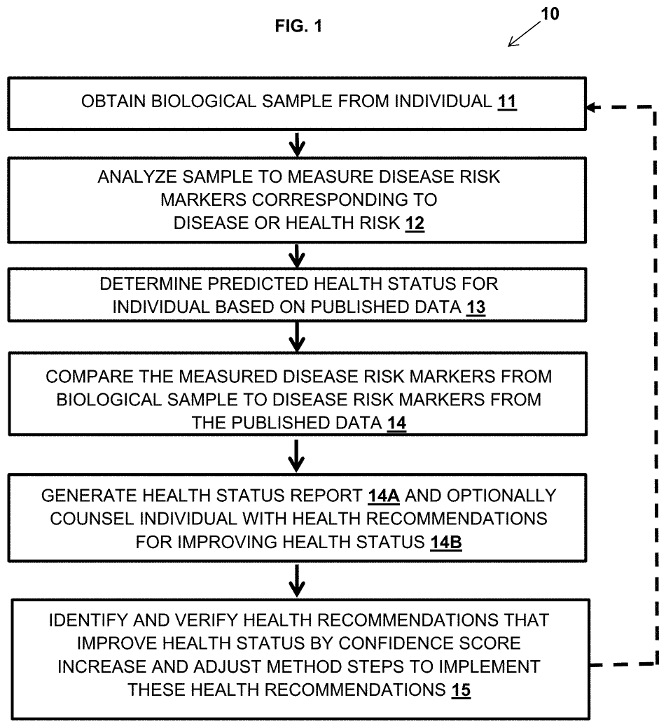

Methods of assessing the health status of an individual can also be described as shown in FIG. 1. FIG. 1 illustrates an example method (10) of assessing the health status of an individual according to an embodiment of the present disclosure. Not all steps illustrated in FIG. 1 are required in the context of the invention, but are provided to illustrate various aspects of thereof. The method (10) comprises obtaining a biological sample from the individual (block 11). The biological sample may be obtained from any source of the individual such as, for example, saliva, blood, urine, amniotic fluid, cerebrospinal fluid or virtually any tissue sample (e.g., from skin, hair, muscle, buccal or conjunctival mucosa, placenta, gastrointestinal tract or other organs). The biological sample is obtained from an individual using any clinically-accepted method. In some embodiments, the biological sample is obtained invasively (e.g., blood draw) in a laboratory or physician's office. While in other embodiments, the sample is obtained non-invasively (e.g., via swabbing or scraping the inside of the mouth). Optionally, the biological sample can be self-collected in the home of the individual using a kit comprising materials for DNA sample collection. An exemplary kit is described in, for example, U.S. Pat. No. 6,291,171, which is hereby incorporated by reference. The collected sample may thereafter be sent directly to the laboratory for analysis.

At block 12, the biological sample is measured to provide measurement data of one or more Disease Risk Markers associated with one or more diseases or health risks that correspond to or impact the quality or condition of the individual's health status. Disease Risk Markers may include Genomic Markers, Proteomic Markers, Metabolomic Markers and Exposomic Markers. Although all four Disease Risk Markers are discussed herein, this is exemplary only, and less than all four Disease Risk Markers may also be utilized with respect to the methods, systems and techniques described herein.

With continued reference to block 12, the biological sample from the individual may be analyzed to determine the presence or absence of the biomarkers. In one aspect, the step of measuring involves determining the presence or absence of one or more polymorphisms in the Genomic Markers, wherein the one or more polymorphisms are associated with the disease or health risk. In one embodiment, such Genomic Markers are selected from the group consisting of genes 1 to 477 in Table 1 (as shown below) or any combination thereof Alternatively, in the method according to embodiments of the present invention, the Genomic Markers are selected from the group consisting of polymorphisms 1 to 477 in Table 1 (as shown below) or any combination thereof. By way of example (and not wishing to be bound by any particular theory), KCNJ11 encodes a potassium inwardly rectifying channel possessing a key role in insulin secretion. An individual with a single nucleotide polymorphism (SNP) in KCNJ11, such as for example rs5215, would have limited insulin secretion function, thereby leading to an increased risk of type 2 diabetes as compared to control subjects that do not possess the SNP (Reference SNP (refSNP) Cluster Report for rs5215; https:/lwww.ncbi.nlm.nih.gov/snp/rs5215). Therefore, this example of the disclosure would benefit those individuals who have the SNP in KCNJ11, thereby requiring possible dietary changes in order to normalize his/her markers and reduce health risks associated with type 2 diabetes.

TABLE 1
Genomic Markers

| No. | Gene | dnSNP ID | Impact on Disease or Health Risk |
|---|---|---|---|
| 1. | EPHX1 | rs2234922 | G allele may affect carbamazepine |
| | | | response. |
| 2. | CARD14 | rs144475004 | Significant increase in risk of psoriasis. |
| 3. | IL12B | rs10045431 | Risk factor for Crohn's disease. |
| 4. | CACNA1C | rs1006737 | A allele is associated with increased risk |
| | | | of bipolar disorder, depression and |
| | | | schizophrenia. |
| 5. | CFH | rs10801555 | Associated with increased risk of age- |
| | | | related macular degeneration. |
| 6. | LBX1 | rs11190870 | Associated with increased risk of |
| | | | adolescent idiopathic scoliosis. |
| 7. | ILB1 | rs1143634 | Associated with variant risk for multiple |
| | | | conditions. |
| 8. | IDUA | rs121965027 | Hurler syndrome mutation(s). |
| 9. | TPH2 | rs1386494 | Associated with increased risk of major |
| | | | depression. |
| 10. | WNT10A | rs146902156 | Tooth agenesis mutation. |
| 11. | IL12A-AS1 | rs17810546 | Associated with increased risk of celiac |
| | | | disease. |
| 12. | CD6 | rs17824933 | G allele is associated with increased risk |
| | | | of multiple sclerosis. |
| 13. | CFTR | rs1800098 | Associated with vas deferens condition. |
| 14. | IL13 | rs20541 | Risk factor for asthma and allergic |
| | | | rhinitis. |
| 15. | STAT2 | rs2066808 | Associated with increased risk of |
| | | | psoriasis. |
| 16. | OPRD1 | rs2236857 | Associated with increased susceptibility |
| | | | to heroin addiction. |
| 17. | OPRD1 | rs2236861 | Associated with increased susceptibility |
| | | | to heroin addiction. |
| 18. | LRP5 | rs312009 | Associated with increased risk of low |
| | | | bone mineral density and osteoporosis. |
| 19. | SLC2A9 | rs3733591 | Associated with increased risk of gout. |
| 20. | SNAP25 | rs3746544 | Associated with ADHD. |
| 21. | VDR | rs3782905 | C allele is associated with increased risk |
| | | | of asthma. |
| 22. | ABCB1 | rs3789243 | T allele is associated with age of onset of |
| | | | Crohn's disease and ulcerative colitis, |
| | | | and is associated with pancolitis in UC |
| | | | patients. |
| 23. | FKBP5 | rs3800373 | G allele is associated with increased risk |
| | | | of major depressive disorder and post- |
| | | | traumatic stress disorder. |
| 24. | CDH9 | rs4307059 | Associated with increased risk of |
| | | | Autism. |
| 25. | KCNJ11 | rs5215 | Associated with increased risk of Type 2 |
| | | | diabetes. |
| 26. | KRT16 | rs57424749 | Associated with pachyonychia |
| | | | congenital Type I mutation. |
| 27. | CHRNA3 | rs6495308 | T allele is associated with smoking |
| | | | quantity. |
| 28. | APOB | rs673548 | Associated with increased levels of |
| | | | serum triglycerides. |
| 29. | FTO | rs7202116 | Associated with increased body mass |
| | | | index. |
| 30. | CETP | rs7499892 | T allele is associated with decreased |
| | | | levels of plasma high density lipoprotein |
| | | | cholesterol. |
| 31. | THADA | rs7578597 | T allele is associated with increased risk |
| | | | of Type 2 diabetes. |
| 32. | TCF7L2 | rs7901695 | T allele is associated with increased risk |
| | | | of Type 2 diabetes and gestational |
| | | | diabetes. |
| 33. | VDR | rs7975232 | C allele is associated with increased risk |
| | | | of childhood asthma and renal cell |
| | | | carcinoma. |
| 34. | LOC100507686 | rs9275596 | Associated with increased risk of |
| | | | developing peanut allergy. |
| 35. | FTO | rs9930506 | Associated with increased BMI. |
| 36. | CFH | rs1061170 | Associated with increased risk for AMD. |
| 37. | SCARB1 | rs5888 | Associated with higher risk for age- |
| | | | related macular degeneration. |
| 38. | TCF4 | rs613872 | Associated with higher risk for Fuchs' |
| | | | dystrophy, a corneal disorder. |
| 39. | CFH | rs1329428 | Associated increased risk for macular |
| | | | degeneration. |
| 40. | TCF7L2 | rs7903146 | Associated with increased risk of type 2 |
| | | | diabetes and colorectal cancer. |
| 41. | LOC101928635 | rs493258 | Associated with increased risk of Age |
| | | | Related Macular Degeneration. |
| 42. | PRNP | rs6107516 | Associated with Creutzfeldt-Jakob |
| | | | disease risk. |
| 43. | CYP2C9 | rs1057910 | CYP2C9*3 carrier associated with |
| | | | reduction in warfarin metabolism. |
| 44. | IFNL3 | rs12979860 | Hepatitis C patients with this genotype |
| | | | respond to treatment. |
| 45. | DRD2 | rs1800497 | Associated with increased frequency of |
| | | | symmetry symptoms in obsessive- |
| | | | compulsive disorder and increased |
| | | | likelihood of responding to bupropion |
| | | | for smoking cessation. |
| 46. | GRIK4 | rs1954787 | Associated with less likely to respond to |
| | | | citalopram. |
| 47. | CYP3A5 | rs776746 | Associated with reduced metabolism of |
| | | | tacrolimus leading to slower clearance |
| | | | from the body. |
| 48. | ABO | rs657152 | Associated with increased risk of |
| | | | coronary artery disease, cardiovascular |
| | | | disease and pancreatic cancer. |
| 49. | DBC1 | rs10984447 | Associated with increased risk of |
| | | | multiple sclerosis. |
| 50. | ITGAM | rs11150610 | Associated with systemic lupus |
| | | | erythematosus. |
| 51. | PADI4 | rs11203366 | Associated with rheumatoid arthritis. |
| 52. | OPRD1 | rs12749204 | Associated with increased risk of |
| | | | cocaine dependence. |
| 53. | ADRB2 | rs1801704 | Associated with variant resistance to |
| | | | malaria and increased risk of asthma. |
| 54. | F11 | rs2036914 | Associated with Increased risk of deep |
| | | | vein thrombosis. |
| 55. | CACNA1C | rs2159100 | T allele is associated with decreased |
| | | | expression of CACNA1C, which may |
| | | | increase schizophrenia risk. |
| 56. | INPP4A | rs2278206 | Associated with increased risk for |
| | | | asthma. |
| 57. | RET | rs2506030 | Risk factor for Hirschsprung disease. |
| 58. | DBH | rs3025382 | Associated with nicotine dependence. |
| 59. | TCF7L2 | rs34872471 | Associated with Type 2 Diabetes |
| | | | Mellitus. |
| 60. | GABBR2 | rs3750344 | Associated with nicotine dependence. |
| 61. | ACE | rs4311 | Associated with increased risk of |
| | | | diabetic nephropathy. |
| 62. | F5 | rs6030 | G allele is associated with increased |
| | | | activated partial thromboplastin time. |
| 63. | CAT | rs769217 | Associated with effect of phthalates on |
| | | | lung function. |
| 64. | CDH13 | rs8055236 | Associated with higher risk for heart |
| | | | disease. |
| 65. | F11 | rs925451 | Associated with increased risk of |
| | | | ischemic stroke. |
| 66. | Intergenic | rs9300039 | Associated with increased risk of T2D. |
| 67. | IL23R | rs11209026 | Associated with higher risk for certain |
| | | | autoimmune diseases. |
| 68. | ADH1B | rs1229984 | Associated with more frequent alcohol |
| | | | consumption. |
| 69. | SLC6A3 | rs27072 | Associated with higher risk of severe |
| | | | alcohol withdrawal. |
| 70. | PITX2, ENPEP | rs10033464 | Associated with increased risk of Atrial |
| | | | Fibrillation and cardioembolic stroke. |
| 71. | IL23R | rs1004819 | Associated with increased risk of |
| | | | Crohn's disease and ankylosing |
| | | | spondylitis. |
| 72. | ASIP | rs1015362 | Associated with increased risk of |
| | | | melanoma. |
| 73. | ATG16L1 | rs10210302 | Associated with increased risk of |
| | | | Crohn's disease |
| 74. | ABCB1 | rs10248420 | Associated with less likely to respond to |
| | | | antidepressants that are substrates of P- |
| | | | glycoprotein. |
| 75. | DPP6 | rs10260404 | Associated with increased risk of |
| | | | developing ALS. |
| 76. | ADRB2 | rs1042713 | Associated with that pediatric inhaler use |
| | | | may make asthma worse. |
| 77. | PGR | rs1042838 | Associated with increased risk of |
| | | | migraine-associated vertigo and |
| | | | endometrial ovarian cancer. |
| 78. | CHRNA4 | rs1044396 | Associated with increased risk of |
| | | | nicotine dependence. |
| 79. | LPA | rs10455872 | Associated with increased risk of |
| | | | coronary heart disease. |
| 80. | GFPT2 | rs10464059 | Associated with increased risk of |
| | | | developing Parkinson's Disease. |
| 81. | KIF1B | rs10492972 | Associated with increased risk for |
| | | | multiple sclerosis. |
| 82. | NOS1AP | rs10494366 | Associated with shorter QT interval. |
| 83. | SLC22A4 | rs1050152 | Associated with increased risk of |
| | | | Crohn's disease. |
| 84. | CYP1B1 | rs1056836 | Associated with increased risk of lung |
| | | | cancer, prostate cancer. |
| 85. | HLA-A | rs1061235 | Associated with increased risk of bad |
| | | | reaction to anti-epileptic carbamazepine. |
| 86. | HLA-DQA1 | rs1071630 | Associated with childhood-onset steroid- |
| | | | sensitive nephrotic syndrome. |
| 87. | AC067751.1 | rs10761659 | Associated with increased risk of |
| | (Intergenic) | | Crohn's disease. |
| 88. | BDNF | rs10767664 | Associated with increased susceptibility |
| | | | to allergic rhinitis and asthma. |
| 89. | CDKN2A/B | rs10811661 | Associated with increased risk of Type-2 |
| | | | diabetes. |
| 90. | MTNR1B | rs10830963 | Associated with increased risk of Type-2 |
| | | | diabetes and gestational diabetes. |
| 91. | BDNF | rs10835210 | Associated with schizophrenia and |
| | | | phobic disorders. |
| 92. | CAT | rs10836235 | Associated with late cardiac damage. |
| 93. | IL23R | rs10889677 | Associated with increased risk of |
| | | | Graves' disease. |
| 94. | MYNN | rs10936599 | C allele is associated with increased risk |
| | | | of ischemic stroke. |
| 95. | CDKN2B-AS1 | rs10965219 | Associated with increased platelet |
| | | | reactivity. |
| 96. | BDNF | rs11030104 | Associated with Alzheimer disease when |
| | | | found in certain haplotypes, and is |
| | | | associated with antipsychotic treatment |
| | | | resistance in schizophrenic patients. |
| 97. | BDNF | rs11030119 | Associated with poor long-term |
| | | | functional outcome after ischemic |
| | | | stroke. |
| 98. | DRD2 | rs11214606 | Associated with olanzapine effect on |
| | | | working memory. |
| 99. | FTO | rs1121980 | Associated with increased risk for |
| | | | obesity. |
| 100. | SLC43A3 | rs11229030 | Associated with increased risk of |
| | | | Crohn's disease. |
| 101. | ABCB1 | rs1128503 | Associated with requiring more |
| | | | methadone during heroin withdrawal. |
| 102. | SERPINF1 | rs1136287 | Associated with increased risk of wet |
| | | | ARMD |
| 103. | GSTP1 | rs1138272 | Associated with increased risk of asthma |
| | | | in relation to air pollution exposure and |
| | | | increased risk of docetaxel-induced |
| | | | nephropathy. |
| 104. | IL23R | rs11465802 | Associated with drug resistance in |
| | | | pulmonary tuberculosis. |
| 105. | LINC01432 | rs1160312 | Associated with increased risk of Male |
| | | | Pattern Baldness. |
| 106. | FTO | rs11642841 | Associated with obesity-related traits. |
| 107. | DGKH | rs1170191 | Associated with bipolar disorder. |
| 108. | DRD1 | rs11746641 | Associated with schizophrenia and |
| | | | smoking abstinence. |
| 109. | AC093106.2 | rs11761231 | Associated with rheumatoid arthritis. |
| | (Intergenic) | | |
| 110. | MC4R | rs11872992 | Associated with higher BMI. |
| 111. | STAT4 | rs11889341 | Associated with rheumatoid arthritis and |
| | | | other inflammatory diseases. |
| 112. | ABCB1 | rs11983225 | Associated with lower responds to |
| | | | antidepressants that are substrates of P- |
| | | | glycoprotein. |
| 113. | ABCB1 | rs1202184 | Associated with pediatric Crohn's |
| | | | disease and major depressive disorder. |
| 114. | CD58 | rs12044852 | Associated with increased risk multiple |
| | | | sclerosis. |
| 115. | CRP | rs1205 | Functional polymorphism of CRP. |
| 116. | FTO | rs12149832 | Associated with obesity and |
| | | | osteoarthritis. |
| 117. | TCF7L2 | rs12255372 | Associated with increased risk of Type-2 |
| | | | diabetes. |
| 118. | LOC105370503 | rs12431733 | Associated with increased risk of |
| | | | developing Parkinson's Disease. |
| 119. | ADIPOQ | rs12495941 | Associated with adiponectin level and |
| | | | increased risk of stroke. |
| 120. | CLEC16A | rs12708716 | Associated with increased risk of Type-1 |
| | | | diabetes. |
| 121. | SIRT1 | rs12778366 | Associated with increased risk of Type 2 |
| | | | diabetes. |
| 122. | DIO2 | rs12885300 | Associated with increased risk of |
| | | | osteoarthritis and bipolar disorder. |
| 123. | MC4R | rs12970134 | Associated with obesity. |
| 124. | SLC2A9 | rs13129697 | Associated with serum uric acid |
| | | | concentration. |
| 125. | SLC6A7 | rs13153971 | Associated with increased risk of |
| | | | Asthma. |
| 126. | SLC30A8 | rs13266634 | Associated with increased risk of Type 2 |
| | | | diabetes. |
| 127. | CFH | rs1329424 | Associated with increased risk of age- |
| | | | related macular degeneration. |
| 128. | CDKN2BAS | rs1333040 | Associated with increased myocardial |
| | | | infarction risk and intracranial aneurysm |
| | | | risk. |
| 129. | FKBP5 | rs1360780 | Associated with increased risk of |
| | | | depression. |
| 130. | LPL | rs13702 | Associated with decreased HDL |
| | | | cholesterol levels. |
| 131. | EDA2R | rs1385699 | Associated with increased risk of male- |
| | | | pattern baldness. |
| 132. | CFH | rs1410996 | Associated with increased risk of age- |
| | | | related macular degeneration. |
| 133. | HTR2C | rs1414334 | Associated with metabolic syndrome |
| | | | when taking antipsychotics. |
| 134. | FTO | rs1421085 | Associated with obesity. |
| 135. | IGF2BP2 | rs1470579 | Associated with increased risk of Type |
| | | | 2 diabetes. |
| 136. | IL23R | rs1495965 | Associated with increased risk of risk for |
| | | | spondylitis. |
| 137. | LPL | rs15285 | Associated with increased triglyceride |
| | | | levels. |
| 138. | FTO | rs1558902 | A allele is associated with higher BMI. |
| 139. | GP6 | rs1613662 | Associated with increased risk of deep |
| | | | vein thrombosis. |
| 140. | COMT | rs165599 | A allele is associated with increased risk |
| | | | of panic disorder and other anxiety- |
| | | | related traits. G allele may be associated |
| | | | with increased risk of schizophrenia. |
| 141. | IL1B | rs16944 | Associated with increased risk of mental |
| | | | illness and osteoarthritis. |
| 142. | GSTP1 | rs1695 | G allele is associated with increased risk |
| | | | of asthma. |
| 143. | SHROOM3 | rs17319721 | Negative associations with kidney |
| | | | function. |
| 144. | MECP2 | rs1734791 | Associated with increased risk of lupus. |
| 145. | ADAD1 | rs17388568 | Associated with increased risk of Type-1 |
| | | | diabetes. |
| 146. | MYRF | rs174537 | Associated with higher LDL-C and |
| | | | cholesterol. |
| 147. | FADS2 | rs174576 | Associated with increased risk of white |
| | | | matter abnormality after preterm birth. |
| 148. | FADS2 | rs174583 | Associated with higher compressive |
| | | | strength index and perinatal depression |
| | | | when found in a certain haplotype. |
| 149. | FADS2 | rs174601 | Associated with increased risk for |
| | | | ischemic stroke when present in a certain |
| | | | haplotype. |
| 150. | AL137026.2 | rs1746048 | Associated with decreased risk for |
| | (Intergenic) | | coronary heart disease. |
| 151. | MIA3 | rs17465637 | Associated with higher risk for |
| | | | myocardial infarction. |
| 152. | WWC1 | rs17551608 | Associated with increased risk of |
| | | | schizophrenia. |
| 153. | BMP4 | rs17563 | C allele is associated with increased risk |
| | | | of otosclerosis and cutaneous melanoma |
| 154. | FMN2 | rs17672135 | Associated with increased risk of |
| | | | coronary artery disease. |
| 155. | FTO | rs17817449 | Associated with increased risk of |
| | | | obesity. |
| 156. | TPH1 | rs1799913 | Associated with increased risk of heroin |
| | | | addiction. |
| 157. | NAT2 | rs1799930 | Associated with increased risk of hearing |
| | | | loss. |
| 158. | OPRM1 | rs1799971 | G allele is associated with increased |
| | | | sensitivity to pain, requirement for |
| | | | higher opioid dosage for pain relief, and |
| | | | increased risk of opioid addiction. May |
| | | | also be associated with increased risk of |
| | | | alcohol dependence and a lower relapse |
| | | | rate when treating alcoholism with |
| | | | naltrexone. |
| 159. | LOC100287329 | rs1800630 | Associated with increased lupus risk. |
| 160. | TNFRSF1A | rs1800693 | Associated with increased in risk for |
| | | | multiple sclerosis. |
| 161. | IL6 | rs1800795 | Associated with increased risk of |
| | | | autoimmune disease. |
| 162. | IL10 | rs1800896 | Associated with increased risk of asthma |
| | | | and susceptibility to infection and |
| | | | increased prostate cancer risk. |
| 163. | CLOCK | rs1801260 | Associated with increased risk of ADHA |
| | | | symptoms. |
| 164. | MC1R | rs1805008 | Associated with increased risk of |
| | | | melanoma. |
| 165. | PLA2G7 | rs1805018 | Associated with atopic asthma. |
| 166. | NOS3 | rs1808593 | Associated with more severe brain |
| | | | damage. |
| 167. | ADIPOQ | rs182052 | Associated with increased risk of Type 2 |
| | | | diabetes and diabetic nephropathy. |
| 168. | CHRM2 | rs1824024 | Associated with increased risk of alcohol |
| | | | dependence and major depressive |
| | | | syndrome when found in a certain |
| | | | haplotype. |
| 169. | CETP | rs183130 | Associated with Lower HDL cholesterol. |
| 170. | TPH2 | rs1843809 | Associated with attention-deficit |
| | | | hyperactivity disorder. |
| 171. | FTO | rs1861868 | Associated with increased BMI and |
| | | | obesity. |
| 172. | IL18 | rs187238 | Associated with increased risk for |
| | | | sudden cardiac death with hvnertension |
| 173. | HIST1H1T | rs198846 | Associated with variations in blood |
| | | | hemoglobin levels. |
| 174. | SLC2A13 | rs1994090 | Associated with increased risk of |
| | | | developing Parkinson's Disease. |
| 175. | NSF | rs199533 | Associated with increased risk of |
| | | | developing Parkinson's Disease. |
| 176. | BDNF | rs2030324 | Associated with internalizing disorders, |
| | | | nicotine dependence and poorer visual |
| | | | cognitive processing in multiple |
| | | | sclerosis. |
| 177. | OPRD1 | rs204076 | Associated with longer hospital stays for |
| | | | infants with neonatal abstinence |
| | | | syndrome. |
| 178. | CHRM2 | rs2061174 | Associated with increased risk of alcohol |
| | | | dependence and major depressive |
| | | | syndrome. |
| 179. | CYP2E1 | rs2070676 | Associated with decreased risk of |
| | | | Parkinson's disease. |
| 180. | LIPC | rs2070895 | A allele is associated with increased high |
| | | | density lipoprotein cholesterol levels and |
| | | | with increased total cholesterol and low |
| | | | density lipoprotein cholesterol. |
| 181. | HLA-C | rs2074488 | Associated with rheumatoid arthritis. |
| 182. | NOD2 | rs2076756 | Associated with increased risk for |
| | | | Crohn's disease. |
| 183. | CFTR | rs213950 | A allele may be associated with |
| | | | increased risk of Type 1 diabetes. |
| 184. | CACNA1C | rs216013 | May influence warfarin dosage. |
| 185. | AL109807.1 | rs2180439 | Associated with increased risk of Male |
| | (Intergenic) | | Pattern Baldness. |
| 186. | LINC01405 | rs2188380 | Associated higher risk of gout. |
| | (Intergenic) | | |
| 187. | IL23R | rs2201841 | Associated with increased risk for |
| | | | Graves' disease. |
| 188. | AL049649.1 | rs2207418 | Associated with increased risk for heart |
| | (Intergenic) | | failure. |
| 189. | HTR2A | rs2224721 | Associated with risk of bipolar disorder |
| | | | when found in a specific haplotype. |
| 190. | ATR | rs2227928 | Poorer response to pancreatic cancer |
| | | | combined treatment. |
| 191. | IGF1R | rs2229765 | A allele is associated with increased risk |
| | | | of Barrett's esophagus, colorectal cancer, |
| | | | and thyroid cancer. |
| 192. | C3 | rs2230199 | Associated with risk of ARMD. |
| 193. | ABCG2 | rs2231137 | Associated with increased risk for |
| | | | ischemic stroke. |
| 194. | ABCB1 | rs2235015 | Associated with reduced likelihood of |
| | | | responding to antidepressants that are |
| | | | substrates of P-glycoprotein. |
| 195. | ABCB1 | rs2235067 | Associated with reduced likelihood of |
| | | | responding to antidepressants that are |
| | | | substrates of P-glycoprotein. |
| 196. | CHRNA4 | rs2236196 | Associated with nicotine dependence. |
| 197. | MET | rs2237717 | Associated with Increased risk of |
| | | | schizophrenia, decreased ability to |
| | | | recognize facial emotion perception, and |
| | | | increased susceptibility to chronic |
| | | | rhinosinusitis. |
| 198. | ATG16L1 | rs2241880 | Associated with increased risk for |
| | | | Crohn's disease. |
| 199. | DRD2 | rs2283265 | G allele associated with increased risk of |
| | | | schizophrenia and increased severity of |
| | | | ADHD. |
| 200. | PPARGC1A | rs2290602 | Associated with increased risk for non- |
| | | | alcoholic fatty liver disease. |
| 201. | MIA3 | rs2291834 | Associated with higher risk for |
| | | | myocardial infarction. |
| 202. | FAAH | rs2295632 | C allele associated with increased risk of |
| | | | early onset, but not adult, extreme |
| | | | obesity. |
| 203. | SPECC1L- | rs2298383 | Associated with increased risk of |
| | ADORA2A | | caffeine-induced anxiety, anxious |
| | | | personality when found in a specific |
| | | | haplotype and increased likelihood of |
| | | | developing rheumatoid nodules in |
| | | | rheumatoid arthritis patients treated with |
| | | | methotrexate when combined with the |
| | | | MTHFR 1298AA genotype. |
| 204. | PTEN | rs2299939 | C allele associated with increased risk of |
| | | | atherosclerotic cerebral infarction when |
| | | | found in a specific haplotype. |
| 205. | MEIS1 | rs2300478 | Associated with risk for developing |
| | | | restless legs syndrome. |
| 206. | TYK2 | rs2304256 | Associated with increased risk of lupus. |
| 207. | GSDMB | rs2305480 | Associated with increased risk of |
| | | | asthma. |
| 208. | EIF3G | rs2305795 | Associated with higher risk of |
| | | | narcolepsy. |
| 209. | CBS | rs234709 | Associated with altered arsenic |
| | | | metabolism. |
| 210. | GAB2 | rs2373115 | May be associated with increased risk of |
| | | | Alzheimer's disease. |
| 211. | HLA-DRB9 | rs2395185 | Associated with increased risk of |
| | | | Ulcerative Colitis. |
| 212. | ADRB2 | rs2400707 | Associated with increased risk of |
| | | | temporomandibular joint disorder |
| | | | (TMD). |
| 213. | FCER1A | rs2427827 | Associated with increased serum IgE |
| | | | levels. |
| 214. | CYP19A1 | rs2470144 | A allele is associated with lower age at |
| | | | menarche and osteoporosis. Associated |
| | | | with decreased risk of colorectal cancer. |
| 215. | AKT1 | rs2494732 | Associated with increased risk of |
| | | | cannabis-associated psychosis. |
| 216. | SLC6A4 | rs25532 | Associated with OCD. |
| 217. | DTNBP1 | rs2619538 | Associated with increased risk of |
| | | | schizophrenia. |
| 218. | DRD1 | rs265981 | G allele is associated with increased risk |
| | | | for autism spectrum disorders. |
| 219. | TERT | rs2736100 | Associated with increased risk of |
| | | | pulmonary fibrosis and glioma |
| | | | development. |
| 220. | SNCA | rs2736990 | Associated with increased risk of |
| | | | developing Parkinson's Disease. |
| 221. | IL12B | rs2853694 | Associated with increased susceptibility |
| | | | to leprosy. |
| 222. | TCF7L2 | rs290481 | Associated with higher 2-h post- |
| | | | challenge glucose and insulin |
| | | | concentration, elevated systolic and |
| | | | diastolic blood pressure, lower waist |
| | | | circumference, and increased steady- |
| | | | state plasma glucose concentration. |
| 223. | WNT16 | rs2908004 | C allele is associated with decreased |
| | | | bone mineral density. |
| 224. | AC062015.1 | rs2943634 | C allele is associated with increased risk |
| | (Intergenic) | | of coronary artery disease. |
| 225. | AC062015.1 | rs2943641 | Associated with increased risk for type 2 |
| | (Intergenic) | | diabetes. |
| 226. | ERAP1 | rs30187 | Associated with higher risk for |
| | | | Ankylosing Spondylitis. |
| 227. | CTLA4 | rs3087243 | Associated with increased risk for auto- |
| | | | immune diseases. |
| 228. | CRP | rs3093059 | C allele is associated with increased |
| | | | serum C-reactive protein levels. |
| 229. | HLA-DRA | rs3135388 | Associated with higher risk of multiple |
| | | | sclerosis. |
| 230. | F2 | rs3136516 | G allele is associated with increased risk |
| | | | of systemic lupus erythematosus. |
| 231. | LPL | rs326 | Associated with lower HDL cholesterol. |
| 232. | AC097478.1 | rs356219 | Associated with increased risk for |
| | (Intergenic) | | Parkinson's disease. |
| 233. | SNAP25 | rs362584 | G allele is associated with neuroticism. |
| 234. | CX3CR1 | rs3732379 | Associated with increased risk of |
| | | | developmental dysplasia of the hip. |
| 235. | CLOCK | rs3736544 | Associated with increased remission to |
| | | | fluvoxamine treatment in patients with |
| | | | major depressive disorder. |
| 236. | HTR2A | rs3742278 | G allele is associated with increased risk |
| | | | of panic disorder. |
| 237. | SPIB | rs3745516 | Associated with increased risk of |
| | | | developing primary biliary cirrhosis. |
| 238. | FTO | rs3751812 | T allele is associated with increased risk |
| | | | of obesity. |
| 239. | F11 | rs3756008 | T allele is associated with increased risk |
| | | | of venous thrombosis. |
| 240. | TRAF1 | rs3761847 | Associated with increased risk of |
| | | | rheumatoid arthritis. |
| 241. | DRD3 | rs3773678 | C allele is associated with nicotine |
| | | | dependence. |
| 242. | TLR3 | rs3775290 | Associated with increased susceptibility |
| | | | to knee osteoarthritis. |
| 243. | TLR3 | rs3775296 | Associated with increased susceptibility |
| | | | to knee osteoarthritis. |
| 244. | ATG16L1 | rs3792109 | T allele may be associated with |
| | | | increased risk of Crohn's disease. |
| 245. | GLCCI1 | rs37973 | Associated with more likely to show less |
| | | | response to inhaled glucocorticoids. |
| 246. | SLC6A4 | rs3813034 | Associated with increased risk of panic |
| | | | disorder. |
| 247. | LOXL1 | rs3825942 | Associated with weakly increased risk of |
| | | | glaucoma. |
| 248. | ABCB1 | rs3842 | T allele associated with increased |
| | | | remission after 8-week antidepressant |
| | | | treatment with desipramine or |
| | | | fluoxetine. |
| 249. | MYH15 | rs3900940 | Associated with increased risk of |
| | | | coronary heart disease. |
| 250. | CRHR1- | rs393152 | Associated with increased risk of both |
| | IT1-CRHR1 | | Parkinson's and Alzheimer's disease. |
| 251. | MIR3681HG | rs4027132 | Associated with increased risk of |
| | (Intergenic) | | developing bipolar disorder. |
| 252. | CNIH2 | rs4073582 | Associated with higher risk for gout. |
| 253. | ABCB1 | rs4148739 | Associated with reduced likelihood of |
| | | | responding to antidepressants that are |
| | | | substrates of P-glycoprotein. |
| 254. | ABCB1 | rs4148740 | Less likely to respond to certain |
| | | | antidepressants. |
| 255. | NEDD4L | rs4149601 | Associated with increased salt |
| | | | sensitivity, increased blood pressure and |
| | | | increased risk of cardiovascular disease. |
| 256. | PPARGC1A | rs4235308 | Protective against Type 2 diabetes. |
| 257. | F11 | rs4253399 | G allele is associated with increased risk |
| | | | of venous thromboembolism . |
| 258. | UMOD | rs4293393 | Associated with increased Risk of CKD |
| | | | for T allele. |
| 259. | ACE | rs4341 | ACE D/D genotype. Associated with |
| | | | obesity and blood pressure. |
| 260. | ACE | rs4343 | ACE D/D genotype. |
| 261. | IGF2BP2 | rs4402960 | Associated with increased risk of type 2 |
| | | | diabetes and gestational diabetes. |
| 262. | VDR | rs4516035 | Associated with increased calcium |
| | | | requirement for vertebral mass accrual. |
| | | | C allele associated with increased risk of |
| | | | melanoma. |
| 263. | DRD1 | rs4532 | T allele is associated with more severe |
| | | | autism spectrum disorder symptoms and |
| | | | with nicotine dependence. |
| 264. | RGS2 | rs4606 | Associated with anxiety related |
| | | | behaviours. |
| 265. | NBPF3 | rs4654748 | C allele is associated with lower vitamin |
| | | | B6 levels in blood. |
| 266. | COMT | rs4680 | Associated with increased risk of breast |
| | | | cancer. |
| 267. | ADORA2A-AS1 | rs4822492 | Associated with increased anxiety in |
| | | | response to caffeine. |
| 268. | SOD2 | rs4880 | T allele is associated with increased risk |
| | | | of cardiomyopathy associated with |
| | | | hereditary hemochromatosis. |
| 269. | CBS | rs4920037 | Associated with variant arsenic |
| | | | metabolism. |
| 270. | PSORS1C1 | rs4959053 | Associated with increased risk of |
| | | | Bechet's disease. |
| 271. | ADD1 | rs4961 | Associated with increased risk of high |
| | | | blood pressure. |
| 272. | LRP5 | rs4988300 | T allele associated with increased risk of |
| | | | obesity. |
| 273. | TLR4 | rs5030728 | Associated with variant effect of |
| | | | saturated fatty acid intake on high |
| | | | density lipoprotein cholesterol. |
| 274. | AGT | rs5051 | Associated with increased risk for |
| | | | hypertension. |
| 275. | APOA2 | rs5082 | Associated with increased energy intake |
| | | | and increased risk of obesity. |
| 276. | OPRM1 | rs510769 | Associated with increased response to |
| | | | amphetamine and increased risk of |
| | | | insomnia. |
| 277. | AGTR1 | rs5182 | Associated with reduced risk of |
| | | | myocardial infarction and increased risk |
| | | | of hypertension. |
| 278. | AGTR1 | rs5186 | Associated with increased risk of |
| | | | hypertension. |
| 279. | GNB3 | rs5443 | Associated with increased risk for |
| | | | several metabolic conditions. |
| 280. | PSRC1 | rs599839 | Associated with increased risk for heart |
| | | | disease. |
| 281. | F5 | rs6028 | C allele may be associated with |
| | | | increased activated partial |
| | | | thromboplastin time. |
| 282. | SLC22A1 | rs622342 | C allele is associated with weaker |
| | | | response and shorter survival on |
| | | | levodopa. |
| 283. | PCSK1 | rs6232 | Higher risk of obesity and insulin |
| | | | sensitivity. |
| 284. | BDNF | rs6265 | Associated with increased risk of |
| | | | anorexia and increased risk of obesity. |
| 285. | HTR2A | rs6313 | Higher risk for Rheumatoid Arthritis. |
| 286. | HTR2A | rs6314 | Higher risk for Rheumatoid Arthritis. |
| 287. | HTR2C | rs6318 | Associated with increased risk of |
| | | | cardiovascular disease and heart attack. |
| 288. | NTF3 | rs6332 | A allele shows trend toward association |
| | | | with adult attention deficit hyperactivity |
| | | | disorder symptoms. |
| 289. | LRP5 | rs634008 | C allele is associated with increased risk |
| | | | of obesity. |
| 290. | SLC6A3 | rs6347 | Associated with Tourette's syndrome. |
| 291. | IL12A-AS1 | rs6441286 | Increased risk of primary biliary |
| | | | cirrhosis. |
| 292. | PXK | rs6445975 | G allele is associated with increased risk |
| | | | of systemic lupus erythematosus. |
| 293. | SLC2A9 | rs6449213 | Associated with higher risk for |
| | | | hyperuricemia. |
| 294. | CELSR2 | rs646776 | A allele is associated with increased risk |
| | | | of coronary artery disease. |
| 295. | FTO | rs6499640 | A allele may be associated with |
| | | | increased risk of metabolic syndrome. |
| 296. | CBS | rs6586282 | Associated with increased risk of severe |
| | | | sepsis. |
| 297. | CHRNA3 | rs660652 | A allele may be associated with |
| | | | increased risk of nicotine dependence. |
| 298. | APOA5 | rs662799 | Associated with increased triglyceride |
| | | | levels and increased risk of coronary |
| | | | heart disease. |
| 299. | STK39 | rs6749447 | Associated with higher blood pressure. |
| 300. | MMP3 | rs679620 | G allele is associated with lower blood |
| | | | pressure. |
| 301. | APOB | rs679899 | G allele is associated with increased risk |
| | | | of chronic kidney disease. |
| 302. | DRD1 | rs686 | A allele is associated with increased risk |
| | | | of autism spectrum disorders, alcohol |
| | | | dependence, and nicotine dependence. |
| 303. | IL7R | rs6897932 | Associated with weakly increased risk of |
| | | | multiple sclerosis. |
| 304. | CDKAL1 | rs6908425 | Associated with increased risk of |
| | | | Crohn's disease. |
| 305. | AL356234.2 | rs6920220 | Associated with increased risk of |
| | (Intergenic) | | rheumatoid arthritis. |
| 306. | HLA-DQA1 | rs6927022 | Associated with increased risk of Type 1 |
| | | | diabetes. |
| 307. | APOB | rs693 | Elevated lipids. |
| 308. | FAM71F1 | rs6971091 | Associated with increased risk for |
| | | | familial obesity. |
| 309. | AGT | rs699 | Associated with increased risk of |
| | | | hypertension. |
| 310. | CYP19A1 | rs700518 | Associated with increased risk of |
| | | | essential hypertension. |
| 311. | BOLL | rs700651 | Associated with increased risk of |
| | | | intracranial aneurysm. |
| 312. | IL2RA | rs706779 | A allele is associated with increased risk |
| | | | of vitiligo. |
| 313. | BDNF | rs7103411 | C allele is associated with comorbid |
| | | | alcohol dependence and tobacco |
| | | | smoking. |
| 314. | HTRA1 | rs714816 | Associated with increased risk of age- |
| | | | related macular degeneration. |
| 315. | TCL1A | rs7158782 | Associated with increased risk of |
| | | | adverse side effects when taking |
| | | | aromatase inhibitors. |
| 316. | FTO | rs7185735 | G allele is associated with increased risk |
| | | | of obesity. |
| 317. | HLA-DRA | rs7192 | Associated with increased risk of |
| | | | allergies, rheumatoid arthritis, systemic |
| | | | lupus erythematosus, psoriasis, and |
| | | | Bechet's disease. |
| 318. | GSDMB | rs7216389 | Associated with increased risk of glioma. |
| 319. | COMT | rs737866 | Associated with increased novelty |
| | | | seeking and earlier age of onset of drug |
| | | | use. |
| 320. | PNPLA3 | rs738409 | Associated with increased liver fat and |
| | | | increased risk of alcoholic liver disease. |
| 321. | SH2B3 | rs739496 | A allele is associated with increased |
| | | | blood pressure and hypertension. |
| 322. | COMT | rs740603 | G allele is associated with cocaine- |
| | | | induced paranoia. A allele is associated |
| | | | with decreased median morphine dose |
| | | | required for treatment of cancer pain. |
| 323. | APOE | rs7412 | Likely to gain weight if taking |
| | | | olanzapine; increased risk for |
| | | | Alzheimer's; increased risk for heart |
| | | | disease. |
| 324. | SLC2A9 | rs7442295 | Associated with higher risk for |
| | | | hyperuricemia. |
| 325. | IL23R | rs7518660 | Associated with increased risk of |
| | | | pulmonary tuberculosis. |
| 326. | STAT4 | rs7574070 | A allele is associated with increased risk |
| | | | of Bechet's disease. |
| 327. | ADIPOQ | rs7627128 | A allele is associated with increased risk |
| | | | of Type 2 diabetes. |
| 328. | CD226 | rs763361 | Associated with increased risk for |
| | | | multiple autoimmune diseases, such as |
| | | | type-1 diabetes. |
| 329. | CDKAL1 | rs7754840 | Associated with increased risk of type 2 |
| | | | diabetes |
| 330. | ABCB1 | rs7787082 | Less likely to respond to certain |
| | | | antidepressants. |
| 331. | SMAD7 | rs78950893 | T allele is associated with increased risk |
| | | | of nonsyndromic cleft lip with or without |
| | | | cleft palate. |
| 332. | TCF7L2 | rs7917983 | T allele is associated with increased risk |
| | | | of hydrochlorothiazide-induced diabetes. |
| 333. | EXOC6 | rs7923837 | Associated risk for type 2 diabetes. |
| 334. | PEMT | rs7946 | T allele is associated with increased risk |
| | | | of non-alcoholic fatty liver disease. |
| 335. | HTR2A | rs7984966 | T allele may be associated with attention |
| | | | deficit hyperactivity disorder |
| | | | phenotypes. |
| 336. | HTR2A | rs7997012 | Less likely to respond to citalopram. |
| 337. | CFH | rs800292 | Associated with higher risk of Age |
| | | | related macular degeneration. |
| 338. | LIPC | rs8034802 | A allele is associated with increased high |
| | | | density lipoprotein cholesterol. |
| 339. | MC4R | rs8087522 | Associated with increased weight gain |
| | | | on clozapine. |
| 340. | ADIPOQ | rs822391 | Associated with possible increased risk |
| | | | of ischemic stroke. C allele is associated |
| | | | with decreased prostate cancer risk. |
| 341. | ADIPOQ | rs822393 | Associated with decreased adiponectin |
| | | | levels and increased risk of nonalcoholic |
| | | | fatty liver disease. |
| 342. | TMPRSS6 | rs855791 | Associated with lower hemoglobin on |
| | | | average. |
| 343. | SNAP25 | rs8636 | Associated with stronger attention deficit |
| | | | hyperactivity disorder symptoms. |
| 344. | PROCR | rs867186 | G allele is associated with increased risk |
| | | | of venous thromboembolism. |
| 345. | STAT4 | rs897200 | Associated with increased expression of |
| | | | STAT4 and increased risk of Bechet's |
| | | | disease. |
| 346. | DHFRP2 | rs9266406 | Associated with increased risk for |
| | | | Bechet's disease. |
| 347. | FMN2 | rs9287237 | G allele is associated with decreased |
| | | | bone mineral density. |
| 348. | IKZF3 | rs9303277 | Associated with increased risk of |
| | | | developing primary biliary cirrhosis. |
| 349. | OPRM1 | rs9479757 | G allele is associated with increased risk |
| | | | of smoking initiation; associated with |
| | | | increased risk of opioid addiction; |
| | | | associated with poor response to |
| | | | oxycodone. |
| 350. | ZNF259 | rs964184 | G allele is associated with increased risk |
| | | | of hypertriglyceridemia. |
| 351. | LINGO1 | rs9652490 | Associated with increased risk of |
| | | | developing Parkinson's Disease. |
| 352. | ADIPOQ | rs9882205 | Associated with lower serum adiponectin |
| | | | levels. |
| 353. | FTO | rs9922619 | T allele is associated with increased risk |
| | | | of severe obesity. |
| 354. | FANCA | rs9926296 | A allele is associated with increased risk |
| | | | of vitiligo. G allele is associated with |
| | | | increased risk of melanoma |
| 355. | ITGAM | rs9937837 | G allele is associated with increased risk |
| | | | of systemic lupus erythematosus and |
| | | | systemic sclerosis. |
| 356. | CETP | rs9939224 | T allele is associated with increased risk |
| | | | of ischemic stroke and decreased high- |
| | | | density lipoprotein levels. |
| 357. | FTO | rs9939609 | Associated with increased risk of obesity |
| | | | and type 2 diabetes. |
| 358. | FTO | rs9940128 | A allele is associated with increased risk |
| | | | of early onset extreme obesity. |
| 359. | FTO | rs9941349 | T allele is associated with increased risk |
| | | | of extreme obesity. |
| 360. | PITX2 | rs2200733 | Associated with decreased risk of Atrial |
| | | | Fibrillation. |
| 361. | LPL | rs320 | G allele is associated with improved |
| | | | lipid profiles. |
| 362. | CHRNA3 | rs578776 | Associated with decreased risk of |
| | | | nicotine dependence. |
| 363. | IL23R | rs7517847 | G allele is associated with decreased risk |
| | | | of Crohn's disease. |
| 364. | SERPING1 | rs2511989 | Associated with decreased age-related |
| | | | macular degeneration risk. |
| 365. | TLR3 | rs3775291 | Associated with decreased risk for dry |
| | | | age related macular degeneration. |
| 366. | SLC2A9 | rs11942223 | Decreased risk for gout and |
| | | | hyperuricemia. |
| 367. | DGKH | rs17646069 | Decreased risk of calcium oxalate stone. |
| 368. | CBS | rs234706 | Associated with reduced risk of cleft lip/palate. |
| 369. | ACVR1B | rs2854464 | Associated with increased muscle |
| | | | strength. |
| 370. | TBX21 | rs4794067 | Associated with risk of lupus and |
| | | | intractable Grave's Disease. |
| 371. | LY9 | rs509749 | Associated with decrease in lupus risk. |
| 372. | NOS3 | rs891512 | Lower blood pressure than those with an |
| | | | A allele. |
| 373. | TP53 | rs1042522 | Associated with increased longevity. |
| 374. | LOC101928635 | rs10468017 | Associated with higher HDL cholesterol. |
| 375. | GJB2 | rs104894396 | Associated with clinvar. |
| 376. | — | rs10505806 | Aspirin use reduces colorectal cancer |
| | | | risk. |
| 377. | CDKN2A, | rs10757278 | Associated with reduced risk for |
| | CDKN2B | | Coronary Heart Disease and reduced risk |
| | | | for Brain Aneurysm and Abdominal |
| | | | Aortic Aneurysm. |
| 378. | HMGA2 | rs10784502 | Higher intracranial volume. |
| 379. | SLCO1B3 | rs11045585 | Associated with lower risk of docetaxel- |
| | | | induced leukopenia/neutropenia. |
| 380. | DRD2 | rs1124493 | Associated with better response to |
| | | | haloperidol. |
| 381. | DRD2 | rs1125394 | Associated with better response to |
| | | | clozapine treatment. |
| 382. | GNB3 | rs1129649 | Associated with decreased salt |
| | | | sensitivity of blood pressure. |
| 383. | TCF7L2 | rs12772424 | Associated with protection against |
| | | | bipolar disorder. |
| 384. | TLR3 | rs13126816 | Associated with increased clearance of |
| | | | hepatitis C virus. |
| 385. | IL23R | rs1343151 | Associated with lower risk for |
| | | | spondylitis. |
| 386. | CETP | rs1532624 | Associated with increased high density |
| | | | lipoprotein cholesterol. |
| 387. | LOC102724001 | rs16973225 | Associated with reduced colorectal |
| | | | cancer risk. |
| 388. | CETP | rs173539 | Associated with increased high-density |
| | | | lipoprotein cholesterol. |
| 389. | FADS2 | rs174577 | Associated with higher compressive |
| | | | strength index. |
| 390. | ERAP1 | rs17482078 | Associated with lower risk for |
| | | | spondylitis. |
| 391. | GCK | rs1799884 | Associated with risk of type 2 diabetes. |
| 392. | PRNP | rs1799990 | Associated with decreased susceptibility |
| | | | to late-onset Alzheimer's disease. |
| 393. | LOC101928635 | rs1800588 | Associated with higher HDL-C levels. |
| 394. | CETP | rs1800775 | Associated with reduced risk of recurrent |
| | | | venous thromboembolism. |
| 395. | CETP | rs1864163 | G allele has been associated with |
| | | | increased high density lipoprotein |
| | | | cholesterol levels. |
| 396. | C1orf127 | rs2003046 | Associated with lower risk of Male |
| | | | Pattern Baldness. |
| 397. | LOC107984314 | rs2060793 | Higher serum levels of vitamin D. |
| 398. | CILP | rs2073711 | Lower risk of Lumbar Disc Disease. |
| 399. | HDAC9 | rs2073963 | Reduced risk of baldness. |
| 400. | BAG3 | rs2234962 | C allele is associated with lower risk of |
| | | | heart failure due to dilated |
| | | | cardiomyopathy. |
| 401. | FCER1A | rs2251746 | Lower IgE levels. |
| 402. | HNF1A | rs2259816 | Associated with decreased levels of |
| | | | circulating C-reactive protein. |
| 403. | ESR1 | rs2273207 | G allele is associated with protection |
| | | | against schizophrenia when found in a |
| | | | specific haplotype. |
| 404. | OPRM1 | rs2281617 | Associated with better response to |
| | | | amphetamine. |
| 405. | MAPT | rs242559 | C allele is associated with decreased risk |
| | | | of Parkinson's disease. |
| 406. | APOC3 | rs2542052 | More prevalent in centenarians, a person |
| | | | who has lived to the age of 100 years. |
| 407. | LIPC | rs261332 | Associated with higher HDL cholesterol. |
| 408. | LIPC | rs261334 | G allele is associated with increased high |
| | | | density lipoprotein cholesterol levels. |
| 409. | WNT16 | rs2707466 | A allele is associated with increased |
| | | | bone mineral density. |
| 410. | AGTR1 | rs275651 | A allele is associated with decreased risk |
| | | | of high-altitude pulmonary edema. |
| 411. | FUT2 | rs281377 | Associated with decreased risk of |
| | | | primary sclerosing cholangitis. |
| 412. | PTPN2 | rs2847281 | Associated with greater reduction in C- |
| | | | reactive protein in rosuvastatin-treated |
| | | | individuals. |
| 413. | AC092110.1 | rs2965667 | Associated with aspirin use reducing |
| | | | colorectal cancer risk. |
| 414. | IL12B | rs3213094 | Associated with risk for psoriasis. |
| 415. | LPL | rs331 | A allele is associated with increased high |
| | | | density lipoprotein cholesterol levels. |
| 416. | SLC39A8 | rs35518360 | Associated with increased risk of |
| | | | schizophrenia. |
| 417. | CHRNA3 | rs3743078 | C allele may be associated with |
| | | | decreased risk of nicotine dependence. |
| 418. | CETP | rs3764261 | Associated with increased levels of high- |
| | | | density lipoprotein (‘good’) cholesterol. |
| 419. | LOC105374476 | rs3775948 | Associated with lower risk for gout. |
| 420. | RELN | rs3914132 | Associated with lower otosclerosis risk. |
| 421. | DGKH | rs4142110 | Associated with decreased risk of |
| | | | calcium oxalate stone. |
| 422. | ABCA1 | rs4149268 | Associated with increased levels of high- |
| | | | density lipoprotein cholesterol. |
| 423. | PALB2 | rs420259 | Associated with reduced risk of Bipolar |
| | | | Disorder. |
| 424. | ACE | rs4359 | Individual's with this gene variant react |
| | | | to the anti-hypertensive drug ramipril |
| | | | quicker than normal. |
| 425. | TPH2 | rs4565946 | T allele is associated with decreased risk |
| | | | of schizophrenia. |
| 426. | SLC6A3 | rs460000 | Increased stimulation in response to |
| | | | amphetamine. |
| 427. | COMT | rs4646312 | Associated with decreased cold pain |
| | | | sensitivity. |
| 428. | PEMT | rs4646404 | C allele is associated with lower waist- |
| | | | to-hip ratio. |
| 429. | LOC102723722 | rs5030656 | Carrier of a CYP2D6*9 allele. |
| 430. | APOB | rs512535 | Associated with attenuation of obesity |
| | | | risk by muscular endurance activity. |
| 431. | G6PC2 | rs573225 | Associated with higher insulinogenic |
| | | | index. |
| 432. | LIPC | rs588136 | C allele associated with increased levels |
| | | | of high density lipoprotein cholesterol. |
| 433. | CETP | rs5882 | Associated with decreased risk of |
| | | | dementia and Alzheimer's disease, but |
| | | | higher levels of high-density lipoprotein |
| | | | cholesterol. |
| 434. | CHRNA5 | rs588765 | T allele is associated with decreased |
| | | | smoking. |
| 435. | COMT | rs6269 | Associated with decreased cold pain |
| | | | sensitivity. |
| 436. | DRD2 | rs6277 | Associated with decreased dopamine |
| | | | signaling. |
| 437. | CLSTN2 | rs6439886 | Associated with increased memory |
| | | | performance. |
| 438. | MIR3184 | rs6505162 | A allele is associated with increased risk |
| | | | of recurrent pregnancy loss. Associated |
| | | | with esophageal cancer and breast |
| | | | cancer. |
| 439. | PON1 | rs662 | Related to stroke and CAD. |
| 440. | ALDH2 | rs671 | Associated with increased risk of |
| | | | esophageal cancer. |
| 441. | SLC2A9 | rs6832439 | A allele is associated with decreased |
| | | | serum uric acid levels. |
| 442. | SLC2A9 | rs6855911 | Associated with decreased risk for gout. |
| 443. | CETP | rs708272 | Associated with reduction in coronary |
| | | | heart disease risk from alcohol |
| | | | consumption. |
| 444. | IRF5 | rs729302 | Associated with decreased risk of |
| | | | developing rheumatoid arthritis. |
| 445. | VDR | rs731236 | T allele is associated with decreased risk |
| | | | of primary biliary cirrhosis while the C |
| | | | allele is associated with decreased risk of |
| | | | autoimmune thyroid disorders while the |
| | | | C allele is associated with increased risk |
| | | | of breast cancer. |
| 446. | SLC2A9 | rs734553 | C allele is associated with decreased |
| | | | serum uric acid levels and protection |
| | | | against gout. |
| 447. | HNF1A | rs735396 | G allele is associated with decreased |
| | | | plasma C-reactive protein levels. |
| 448. | CYP1A2 | rs762551 | A allele is associated with increase in |
| | | | breast cancer risk. |
| 449. | FKBP5 | rs7757037 | Associated with decreased risk for |
| | | | bipolar disorder. |
| 450. | FAM3C | rs7776725 | Associated with higher bone mineral |
| | | | density. |
| 451. | DBH | rs77905 | T allele is associated with increased |
| | | | effectiveness of nicotine-replacement |
| | | | therapy. |
| 452. | VKORC1 | rs8050894 | Requires lower doses of warfarin. |
| 453. | MAPT | rs8070723 | Associated with reduced risk of |
| | | | developing progressive supranuclear |
| | | | palsy. |
| 454. | KL | rs9536314 | Associated with increased longevity, |
| | | | although this evidence is preliminary. |
| 455. | FTO | rs9936385 | Associated with increased risk of |
| | | | obesity. |
| 456. | CCL11 | rs1129844 | Delay in onset of early-onset |
| | | | Alzheimer's. |
| 457. | BRCA2 | rs1799943 | A allele may be associated with |
| | | | decreased risk of cardiovascular disease. |
| 458. | G6PD | rs1050829 | G6PD Type B. Associated with |
| | | | protection against oxidative damage. |
| 459. | FUT2 | rs492602 | Associated with regulation of proper |
| | | | vitamin B12 absorption and plasma |
| | | | levels and dysfunction may lead to |
| | | | vitamin B12 deficiency. |
| 460. | TYR | rs1042602 | Associated with less freckling. |
| 461. | RPL6P5 | rs10427255 | Associated with increased odds of photic |
| | | | sneeze reflex. |
| 462. | CYP2C9 | rs1057911 | Carrier of one CYP2C9_50298A > T |
| | | | allele. |
| 463. | MC4R | rs10871777 | Associated with higher BMI. |
| 464. | TCHH | rs11803731 | Associated with curlier hair. |
| 465. | IRF4 | rs12203592 | Associated with slightly lighter hair and |
| | | | eye color, less tanning ability. |
| 466. | HERC2 | rs12913832 | Associated with brown eye color. |
| 467. | ABCC11 | rs17822931 | Associated with normal body odor. |
| 468. | TGFB1 | rs1800469 | Associated with higher TGF-Î²1 levels. |
| 469. | CYP1A1 | rs2470893 | A allele is associated with increased |
| | | | coffee consumption. |
| 470. | CYP19A1 | rs3751599 | Associated with height. |
| 471. | OR2M7 | rs4481887 | Associated with ability to smell |
| | | | asparagus metabolites in urine. |
| 472. | LCE3E | rs499697 | Associated with straighter hair. |
| 473. | G6PC2 | rs560887 | Associated with slightly higher fasting |
| | | | plasma glucose levels. |
| 474. | WNT10A | rs7349332 | Associated with straighter hair. |
| 475. | ABO | rs8176719 | Likely to be of blood type A or B. |
| 476. | FADS2 | rs968567 | A allele is associated with increased |
| | | | delta-6 desaturase activity and higher |
| | | | ALA and lower EPA and DPA levels. |
| 477. | PKD1L3 | rs9938025 | Higher odds of dry earwax. |

The presence or absence of polymorphisms is determined using any suitable method. The method by which detection of polymorphism is carried out is not critical. For example, occurrence of the polymorphisms can be detected by a method including, but not limited to, hybridization, restriction fragment length analysis, invader assay, gene chip hybridization assays, oligonucleotide litigation assay, ligation rolling circle amplification, 5′ nuclease assay, polymerase proofreading methods, allele specific PCR, matrix assisted laser desorption ionization time of flight (MALDI-TOF) mass spectroscopy, ligase chain reaction assay, enzyme-amplified electronic transduction, single base pair extension assay, reducing sequence data and sequence analysis.

The polynucleotide material used in the analysis can be DNA (including, e.g., cDNA) or RNA (including, e.g., mRNA), as appropriate. Optionally, the RNA or DNA is amplified by polymerase chain reaction (PCR) prior to hybridization or sequence analysis. For hybridization, the polynucleotide sample exposed to oligonucleotides specific for region of the sequence associated with the polymorphism, optionally immobilized on a substrate (e.g., an array or microarray). Selection of one or more suitable probes specific for a locus of interest and selection of a suitable hybridization condition or PCR condition, are within the ordinary skill of scientists who work with nucleic acids.

While genomic markers are described above, in a further embodiment, other biomarkers including Proteomic Markers, Metabolomic Markers and Exposomic Markers can be analyzed using the methods described herein. Examples of such biomarkers that can be measured in a urine sample are provided in Table 2:

TABLE 2
Biomarkers from Urine

| | | Precursors/pathways |
|---|---|---|
| No. | Chemical Name | (if applicable) |
| 1. | 2-Methylhippuric acid | glycine, |
| | | benzoic acid |
| 2. | 2-OH-Glutaric acid | — |
| 3. | 3 -Deoxyglucosone | — |
| 4. | 4-Ethylphenyl sulphate | — |
| 5. | ADMA (Asymmetric dimethylarginine) | — |
| 6. | SDMA (Symmetric dimethylarginine) | — |
| 7. | Argininic acid | — |
| 8. | Benzoic acid | — |
| 9. | β-alanine | — |
| 10. | β-Hydroxybutyric acid | — |
| 11. | Betaine | — |
| 12. | cis-4-OH-Pro (cis-4-hydroxy-proline) | — |
| 13. | Choline | — |
| 14. | Citric acid | — |
| 15. | CMPF (3-carboxy-4-methyl-5- | — |
| | propyl-2-furanpropanoic acid) | |
| 16. | Creatine | — |
| 17. | Creatinine | — |
| 18. | Diacetylspermine | arginine, |
| | | ornithine and |
| | | methionine |
| 19. | Dimethyl-glycine | — |
| 20. | DOPA (3,4-dihydroxyphenylalanine) | — |
| 21. | Dopamine | — |
| 22. | Fumaric acid | — |
| 23. | Glutaric acid | — |
| 24. | Glyoxal | — |
| 25. | Guanidinopropionic acid | — |
| 26. | Hippuric acid | glycine, |
| | | benzoic acid |
| 27. | Histamine | — |
| 28. | Homocysteine | — |
| 29. | Homovanillic acid | catecholamine |
| 30. | HPHPA (3-(3-Hydroxyphenyl)- | phenylalanine |
| | 3-hydroxypropanoic acid) | |
| 31. | Indole acetic acid | tryptophan |
| 32. | Indoxyl glucoside | tryptophan |
| 33. | Indoxyl glucuronide | tryptophan |
| 34. | Indoxyl sulfate | tryptophan |
| 35. | Kynurenic acid | tryptophan |
| 36. | Kynurenine | tryptophan |
| 37. | Lactic acid | — |
| 38. | Methylhistidine | histidine |
| 39. | Methylmalonic acid | TCA cycle |
| 40. | N-Acetyl-Ala | — |
| 41. | N-Acetyl-Arg | — |
| 42. | N-Acetyl-Asn | — |
| 43. | N-Acetyl-Asp | — |
| 44. | N-Acetyl-Gln | — |
| 45. | N-Acetyl-Glu | — |
| 46. | N-Acetyl-Gly | — |
| 47. | N-Acetyl-His | — |
| 48. | N-Acetyl-Leu/Ile | — |
| 49. | N-Acetyl-Met | — |
| 50. | N-Acetyl-Pro | — |
| 51. | N-Acetyl-Ser | — |
| 52. | N-Acetyl-Trp | — |
| 53. | N-Acetyl-Tyr | — |
| 54. | N-α-Acetyl-Lys | — |
| 55. | Nitro-Tyr (Nitro-tyrosine) | — |
| 56. | N-Methyl-Asp (N-Methyl-aspartic acid) | — |
| 57. | N-ε-Acetyl-Lys | — |
| 58. | Orotic acid | — |
| 59. | Oxalic acid | — |
| 60. | Putrescine | Arginine and |
| | | ornithine |
| 61. | Phe (Phenylalanine) | — |
| 62. | p-Cresol sulfate | tyrosine |
| 63. | p-Hydroxyhippuric acid | glycine and |
| | | benzoic acid |
| 64. | p-Hydroxyphenylacetic acid | — |
| 65. | Pyruvic acid | — |
| 66. | Quinaldic acid | tryptophan |
| 67. | Quinoline 4 carboxylic acid | tryptophan |
| 68. | Quinolinic acid | tryptophan |
| 69. | Sarcosine | glycine |
| 70. | Serotonin | tryptophan |
| 71. | Spermidine | arginine, |
| | | ornithine and |
| | | methionine |
| 72. | Spermine | arginine, |
| | | ornithine and |
| | | methionine |
| 73. | Succinic acid | — |
| 74. | trans-4-OH-Pro (Trans-4-hydroxy-proline) | — |
| 75. | total-Butyric acid | — |
| 76. | Thymine | — |
| 77. | TMAO (Trimethylamine N-oxide) | choline, betaine |
| | | and carnitine |
| 78. | Trp (Tryptophan) | — |
| 79. | Tyr (Tyrosine) | — |
| 80. | Tyramine | — |
| 81. | Uracil | — |
| 82. | Uric acid | — |
| 83. | Uridine | — |
| 84. | Xanthine | — |
| 85. | Xanthosine | — |

Without being limiting, levels of one or more of the biomarkers in Table 2 may be indicative of the presence of a particular disease condition or risk of developing such condition. By way of example, and without being limiting, autism and/or chronic kidney disease may be correlated with the biomarkers Indoxyl sulfate (Dieme et al., J Proteome Res, 2015 Dec. 4; 14(12):5273-82; and Leong et al., J Proteome Res, 2015 Dec. 4; 14(12):5273-82) and p-Cresol sulfate (Gabriele et al., J Proteome Res, 2015 Dec. 4; 14(12):5273-82 and J Proteome Res, 2015 Dec. 4; 14(12):5273-82).

Referring again to block 12, the biological sample from the individual may be analyzed to determine the levels of the biomarkers in the biological sample. In another aspect, the step of measuring preferably involves comparing levels in the biological sample of the Proteomic Markers, the Metabolic Markers, the Exposomic Markers or a combination thereof with levels of the corresponding markers from the published data from samples from individuals that have the disease or health risk, wherein the levels are associated with the disease or health risk. In other words, the levels of the biomarkers in the biological sample are compared against the levels of the biomarkers in the database that have correlated bodily functions with diseases or health risks to identify biomarkers that are outside of the optimal range.

Preferably, the method according to the present invention where the Exposomic Markers are selected from the group consisting of: vitamin, amino acid, inorganic compound, biogenic amine, organic acid, amine oxide, hydrocarbon derivative and a combination thereof. In one aspect, the vitamin is preferably selected from the group consisting of: vitamin A, vitamin B3-amide, vitamin B6, vitamin B1, calcidiol, vitamin D2, vitamin B7, vitamin B5, vitamin B2 and a combination thereof In another aspect, the amino acid is preferably selected from the group consisting of: branched chain amino acid, aromatic amino acid, aliphatic amino acid, polar side chain amino acid, acidic and basic amino acid, and unique amino acid preferably glycine and proline, and a combination thereof. In yet another aspect, the inorganic compound is preferably selected from the group consisting of: copper, iron, sodium, calcium, potassium, phosphorus, magnesium, strontium, rubidium, antimony, selenium, cesium, zinc, barium and a combination thereof. In yet another aspect, the biogenic amine is preferably selected from the group consisting of: trans-OH-proline, acetyl-ornithine, alpha-aminoadipic acid, beta-alanine, taurine, carnosine, methylhistidine and a combination thereof. In yet another aspect, the organic acid is preferably selected from the group consisting of: hippuric acid, 3-(3-hydroxyphenyl)-3-hydroxypropionic acid, 5-hydroxyindole-3-acetic acid, sarcosine, hydroxyphenylacetic acid and a combination thereof. In yet another aspect, the amine oxide is preferably trimethylamine N-oxide. In yet another aspect, the hydrocarbon derivative is preferably trigonelline.

According to one embodiment, the Metabolomic Markers (also referred to herein as “Metabolic Markers”) are selected from the group consisting of: acylcarnitine, biogenic amine, lysophospholipid, glycerophospholipid, sphingolipid, organic acid, amino acid, sugar, hydrocarbon derivative and a combination thereof. In one aspect, the Metabolic Markers are the acylcarnitines preferably selected from the group consisting of: long chain acylcarnitines, medium chain acylcarnitines, and short chain acylcarnitines and a combination thereof. In yet another aspect, the Metabolic Markers are preferably the biogenic amines selected from the group consisting of: creatines, kynurenines, methionine-sulfoxides, spermidines, spermines, asymmetric dimethylarginines, putrescines, serotonins and a combination thereof. In yet another aspect, the Metabolic Markers are preferably lysophosphatidylcholines. In yet another aspect, the Metabolic Markers are preferably glycerophospholipids. In yet another aspect, the Metabolic Markers are sphingolipids preferably selected from the group consisting of: sphingolipids, hydroxy fatty acid sphingomyelins and a combination thereof. In yet another aspect, the Metabolic Markers are organic acids preferably selected from the group consisting of: short chain fatty acids, medium chain fatty acids, and long chain fatty acids and a combination thereof. In yet another aspect, the Metabolic Markers are amino acids preferably selected from the group consisting of: betaines, creatines, citric acids and a combination thereof. In yet another aspect, the Metabolic Markers are preferably glucose. In yet another aspect, the Metabolic Markers are hydrocarbon derivatives preferably selected from the group consisting of: lactic acids, pyruvic acids, succinic acids and a combination thereof.

According to another embodiment, the Proteomic Markers for use in certain embodiments of the disclosed method are selected from the group consisting of: blood clotting protein, cell adhesion protein, immune response protein, transport protein, enzyme, hormone-like protein and a combination thereof. In one aspect, the blood clotting protein is preferably selected from the group consisting of: Protein Z-dependent protease inhibitor, coagulation factor proteins, Antithrombin-III, Plasma serine protease inhibitor, Plasminogen, Prothrombin, Carboxypeptidase B2, Kininogen-1, Vitamin K-dependent protein S, Alpha-2-antiplasmin, Fibrinogen gamma chain, Tetranectin, Heparin cofactor 2, Fibrinogen beta chain, Fibrinogen alpha chain, Vitamin K-dependent protein Z, Alpha-2-macroglobulin, Endothelial protein C receptor, von Willebrand Factor and a combination thereof. In another aspect, the cell adhesion protein is preferably selected from the group consisting of: Inter-alpha-trypsin inhibitor heavy chain H1, Cartilage acidic protein 1, Inter-alpha-trypsin inhibitor heavy chain H4, Proteoglycan 4, Fibronectin, Vitronectin, Attractin, Intercellular adhesion molecule 1, Lumican, Galectin-3-binding protein, Cadherin-5, Leucine-rich alpha-2-glycoprotein 1, Tenascin, Vasorin, Fibulin-1, Probable G-protein coupled receptor 116, L-selectin, Thrombospondin-1 and a combination thereof. In yet another aspect, the immune response protein is preferably selected from the group consisting of: Mannose-binding protein C, Complement component proteins, Ficolin-2, Kallistatin, Plastin-2, Ig mu chain C region, Protein AMBP, CD44 antigen, Ficolin-3, IgGFc-binding protein, Mannan-binding lectin serine protease 2, Serum amyloid A-1 protein, Beta-2-microglobulin, Protein S100-A9, C-reactive protein and a combination thereof. In yet another aspect, the transport protein is preferably selected from the group consisting of: Apolipoproteins, Alpha-1-acid glycoprotein 1, Serum albumin, Retinol-binding protein 4, Hormone-binding globulins, Serotransferrin, Clusterin, Beta-2-glycoprotein 1, Phospholipid transfer protein, Beta-2-glycoprotein 1, Phospholipid transfer protein, Hemopexin, Inter-alpha-trypsin inhibitor heavy chain H2, Gelsolin, Transthyretin, Afamin, Histidine-rich glycoprotein, Serum amyloid A-4 protein, Lipopolysaccharide-binding protein, Haptoglobin, Ceruloplasmin, Vitamin D-binding protein, Hemoglobin subunit alpha 1 and a combination thereof. In yet another aspect, the enzyme is preferably selected from the group consisting of: Phosphatidylinositol-glycan-specific phospholipase D, Carboxypeptidase N subunit 2, Serum paraoxonase/arylesterase 1, Biotinidase, Glutathione peroxidase 3, Carboxypeptidase N catalytic chain, Cholinesterase, Xaa-Pro dipeptidase, Carbonic anhydrase 1, Lysozyme C, Peroxiredoxin-2, Beta-Ala-His dipeptidase and a combination thereof. In yet another aspect, the hormone-like protein is preferably selected from the group consisting of: Extracellular matrix protein 1, Alpha-2-HS-glycoprotein, Angiogenin, Insulin-like growth factor-binding protein complex acid labile subunit, Fetuin-B, Adipocyte plasma membrane-associated protein, Pigment epithelium-derived factor, Zinc-alpha-2-glycoprotein, Angiotensinogen, Insulin-like growth factor-binding protein 3, Insulin-like growth factor-binding protein 2 and a combination thereof.

The level of the biomarkers is determined using any suitable method. That is, the method by which measurement of the level of the biomarkers is not critical. For example, biomarker levels may be measured using a variety of methods, including but not limited to, mass spectrometry, liquid chromatography, enzyme-linked immunosorbent assay (ELISA), etc. In one aspect, the current platform uses a combination of multiple reaction monitoring mass spectrometry, high performance liquid chromatography, and liquid chromatography-mass spectrometry to achieve the most accurate, quantifiable, and reliably consistent biomarker levels results.

At block 13, a predicted health status is determined based on the measurement data of the individual. For example, the measurement data of the individual may be inputted into or operated on by a predictive equation to determine the predicted health status. In some aspects, the predictive equation (described in more detail below) is based on the respective strengths of correlation of the published data on the Disease Risk Markers to the respective diseases or health risks. The predictive equation is determined by a multivariate regression analysis of published data of human subjects that have the disease or health risk.

In some embodiments, the predicted health status of the individual corresponds to the risk of developing one or more diseases or health risks over the lifetime of the individual (or at least over an extended period of time such as, for example, at least two months, at least four months, at least six months, at least a year, at least two years, at least five years, at least a decade, at least two decades, at least four decades or at least five decades). Therefore, it is an effective method and system to generate information for monitoring of future health status changes of the individual. Indeed, it is possible that the correlation between certain of the biomarkers and the disease or health risk is stronger in aged individuals. In various aspects, the predicted health status is representative, or a quantitative indication, of an individual's overall health (at least with respect to the Disease Risk Markers analyzed) over an extended period of time.

The results of the measurement are then compared to disease risk markers from published data associated with the disease or health risk (block 14). As an illustrative but non-limiting example, a bodily fluid sample (e.g., blood sample) obtained from the individual is analyzed to determine the level of 4 biomarkers associated with inflammation, specifically, glycine (low), alpha-Aminoadipic acid (low), Alpha-1-acid glycoprotein 1 (high) and Mannose-binding protein C (high). Each Disease Risk Marker's level is reflected by a respective weighting (e.g., low, high or optimal) of its contribution to the disease or health risk (i.e., chronic joint pain experienced by the individual). The predicted health status includes the weightings corresponding to each Disease Risk Marker's level in the biological sample of the individual.

A predicted health status also can be considered as a measure of an individual's “predicted” health, and, as such, provides useful information in counseling an individual on actionable measures for possible improvements in health status. A health status report is generated based on the predicted health status (block 14A) and is representative of the individual having the disease or health risk or risk of developing thereof. Optionally, a predicted health status can also be used to personalize health recommendations, including systems and methods of counseling an individual based, in part, on information gathered about the individual's physiology and environmental influences for improving his/her health status (block 14B). Both of the health status report and health recommendations can be displayed to the individuals via a web-based or mobile application platform.

In an embodiment, a respective predicted health status is determined for each of the disease or health risk. For example, a method of calculating a predicted health status is to take published data with subjects having the disease or health risk and analyze each of them for the correlation to each of the Disease Risk Marker. With that data, it is possible to then formulate a predictive equation for each Disease Risk Marker which correlates to prevalence of each biomarker to each of the disease or health risk, and then applied to the measurement data.

These disease or health-risk specific predicted health status are referred herein as “respective predictive health status” and each may be representative or indicative of a risk of having the respective disease or health risk or developing the respective disease or health risk at a later period of time or may be representative or indicative of a maximum degree of development of the respective disease or health risk in the individual. For example, a first respective predictive health status may operate on genetic (e.g., KCNJ11) to determine a predicted increase risk of type-2 diabetes, and a second respective predictive health status may operate on lower metabolic biomarker (e.g., creatine) to determine a predicted increased pre-diabetic risk. As a result, the method of the present invention provides for a comprehensive overview of the individual's health status.

According to one aspect of the present disclosure, the predictive equation is determined based on published research data of human subjects having the disease or health risk. Each respective predictive equation includes a confidence score corresponding to a correlation of a particular Disease Risk Marker to the disease or health risk. In certain aspects, the confidence score is based on the strength of predictiveness of the published data used to determine the likelihood of having or at risk of developing the disease or health risk. In one embodiment, the confidence score is an indication of the likelihood that the published data has reproducible results, and wherein the confidence score is weighted based on a comparison of a number of citations received by the published data and a number of references cited by the published data. In other words, the confidence score is a reflection of the reproducibility of the published data. The confidence score is based on the output from a return-on-bibliography (ROB) score calculation, which is the scoring metric developed by the inventors to evaluate the reproducibility of published research information. For example, the ROB score is defined as follows:

$$ROB Score = \frac{Number of Citations}{[ 1 + Number of References Cited ]}$$

The calculation of the ROB score includes two parts: (i) the numerator, which is the number of times that the publication has been cited by other papers in scientific literature, and (ii) the denominator, which is the number of times that the publication has reference other papers within the publication. It is worth noting that the denominator includes the addition of 1 because it is possible, although very rare, that a publication has not cited any references within the publication, and this prevents division by “0”. It is also worth noting that the denominator for a particular publication is fixed once the paper is published and it may grow at different rates depending on the volume of new citations over time. Therefore, it is important to calculate the ROB score for the original publication.

The number of citations received may be captured for previous years all the way up to the year of publication, which allows for a timeline of citation performance thus far. Alternatively, the ROB score may be specified for a particular period such as, for example, the current year as it applies to a specific publication. A ROB score for a particular period, for example, in the year 2019, gives the total performance of all the publications up to that period. For example, the ROB score in 2019 of a publication published in 2008 would count the corresponding papers published from 2008 until 2019 by the publication, which is given by:

$$ROB {Score}_{2019} = \frac{Total Number of Citations Received until 2019}{[ 1 + Number of References Cited ]}$$

When the ROB score of a publication is specified for a particular year, the denominator is also fixed. As a result, it may be concluded that the ROB score of a particular publication may increase but will never decrease over time and that the rate of increase in ROB scores can be different between publications and be used to track performance. A higher ROB score of a particular publication up to the current year is directly proportional to the overall performance of the publication and therefore is indicative of its strength of evidence (i.e., reproducibility) in research literature.

To facilitate the calculation of citation and ROB scores for each publication, Applicant has developed a python script to query publication databases (e.g., Google Scholar) and output both numerator (number of citations) and denominator (number of references) for each identified publication for each Disease Risk Marker. For example, the python script may follow the format:

import json
import pandas as pd
from Bio import Entrez
import xml.etree.ElementTree as ET
import scholarly
## Change Source file here:
filename =“../data/references_test.csv”
def hasReferenceInfo(article):

for item in article[‘MedlineCitation’]:

if item == ‘CommentsCorrectionsList’:

return True

return False

def hasDOIInfo(article):

for item in article[‘PubmedData’][‘ArticleIdList’]:

if item.attributes[‘IdType’] == ‘doi’:

return True

return False

def parseReferences(article):

##

==========================================================
============================

## I am assuming the list of articles under “CommentsCorrectionsList” are the

references

##

==========================================================
============================

referenceList = [ ]
if hasReferenceInfo(article):

referenceList = [x[‘PMID’].decode( ) for x in

article[‘MedlineCitation’][‘CommentsCorrectionsList’] if x.attributes[‘RefType’]
== ‘Cites']

return referenceList

def parseDOI(article):

##

==========================================================
============================

## Parsing the DOI to be used with Google Scholar search library.
##

==========================================================
============================

doi = ‘-’
if hasDOIInfo(article):

article_ids = article[‘PubmedData’][‘ArticleIdLisf]

for item in article_ids:

if item.attributes[‘IdType’] == ‘doi’:

doi=item

return doi

def runPubMed(row):

pmid = row.pmid
handle = Entrez.efetch(db=‘pubmed’, id=pmid, retmode=‘xml’)
result = Entrez.read(handle)
article = result[‘PubmedArticle’][0]
refs = parseReferences(article)
doi = parseDOI(article)
row[‘doi’] = doi
row[‘pubmed_reference_count’] = len(refs)
row[‘pubmed_references'] = “, ”.join(refs)
return row

def runGoogleScholarCitations(row):

if row.doi != ‘-’:

search_query = scholarly.search_pubs_query(row.doi)
obj = next(search_query)
return obj.citedby

return ‘-’

df = pd.read_csv(filename)
df.head( )
##
==========================================================
============================
## Run PubMed for a list of PMIDs
##
==========================================================
============================
print (“running PubMed search for dois and reference count...”)
df = df.apply(runPubMed, axis=1, reduce=False)
print (“done running PubMed search”)
##
==========================================================
============================
## Run GoogleScholar for a list of DOIs
##
==========================================================
============================
print (“running Google Scholar search for citations count... (this one is slow)”)
df[‘scholar_citation_count’] = df.apply(runGoogleScholarCitations, axis=1,
reduce=False)
print (“done with Google Scholar search”)
## Save results!
df.to_csv(“../data/references_exported.csv”)

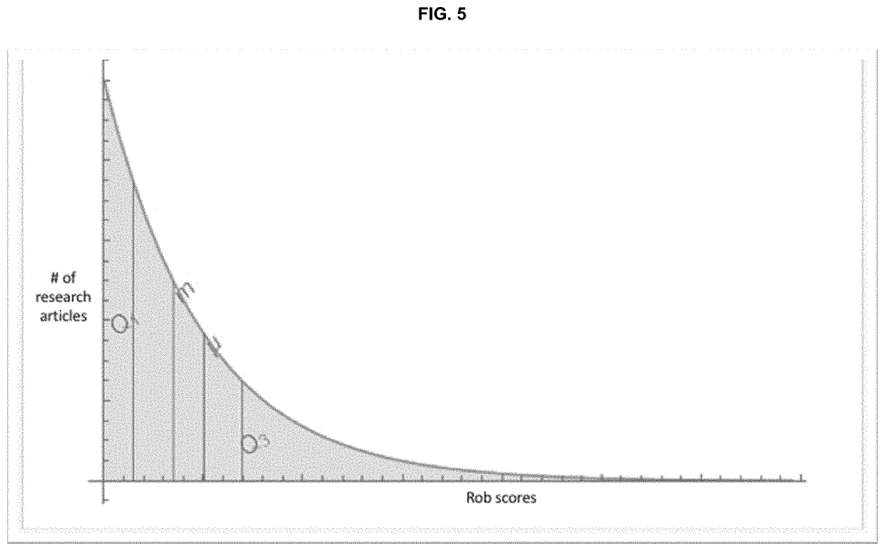

The output from the ROB score calculation may range from 1 to hundreds of thousands, which will not be readily useful or comprehensible to the individual. Therefore, Applicant has formulated the confidence score (ranging in scale from 1 to 4) to simply represent the correlation of the biomarkers to the disease or health risk. In order to determine the confidence score, all of the ROB scores are plotted into a distribution graph and separated into 4 quartiles (as shown in FIG. 5). The quartiles-separated ROB scores are grouped into: (i) first quartile; (ii) second quartile, (iii) third quartile; and (iii) fourth quartile. Specifically, the first quartile represents minimum ROB scores to ROB scores that are at most 25% of the total ROB score ranges, and is defined as having a confidence score of 1. This is typically the minimal threshold required to ensure reliability of the biomarker to disease association. The second quartile represents ROB scores that are greater than 25% of the total ROB score ranges to the median ROB score, and is defined as having a confidence score of 2. The third quartile represents ROB scores that are greater than the median ROB score to ROB scores that are at most 75% of the total ROB score ranges, and is defined as having a confidence score of 3. The fourth quartile represents ROB scores that are greater than 75% of the total ROB score ranges, and is defined as having a confidence score of 4. A summary of the confidence score is provided in the table below.

TABLE 3
Correlation between ROB Score and Confidence Score

| | ROB | Confidence |
|---|---|---|
| | Score | Score |
| First Quartile | Min. to 25% of total ROB | 1 |
| | Score Ranges | |
| Second Quartile | >25% of total ROB Score | 2 |
| | Ranges to Median | |
| Third Quartile | >Median to 75% of total | 3 |
| | ROB Score Ranges | |
| Fourth Quartile | >75% of total ROB Score | 4 |
| | Ranges | |

It will be readily understood that the confidence score may be represented by a score from 1 to 4, with 1 being the values grouped together as the lower confidence (i.e., lower ROB scores) and reflecting lower strength of published evidence as to reproducibility. Conversely, values grouped together near the top end are defined as the highest level of confidence with a confidence score of 4 (i.e., higher ROB scores) and indicating higher strength of published evidence as to reproducibility. Put another way, the confidence score refers to the strength of evidence from the published literature or also known as the “publication evidence score”.

In one aspect of the disclosure, the predictive equation is determined based on the published data. Each respective predictive equation may include a confidence score corresponding to a correlation of a particular Disease Risk Marker to the disease or health risk. As described herein, the value of each confidence score may be determined by a multivariate regression analysis of a plurality of measurements of the Disease Risk Markers of the subjects from the published data. Preferably, the confidence score is weighted based on a comparison of a number of citations received by the published data and a number of references cited by the published data.

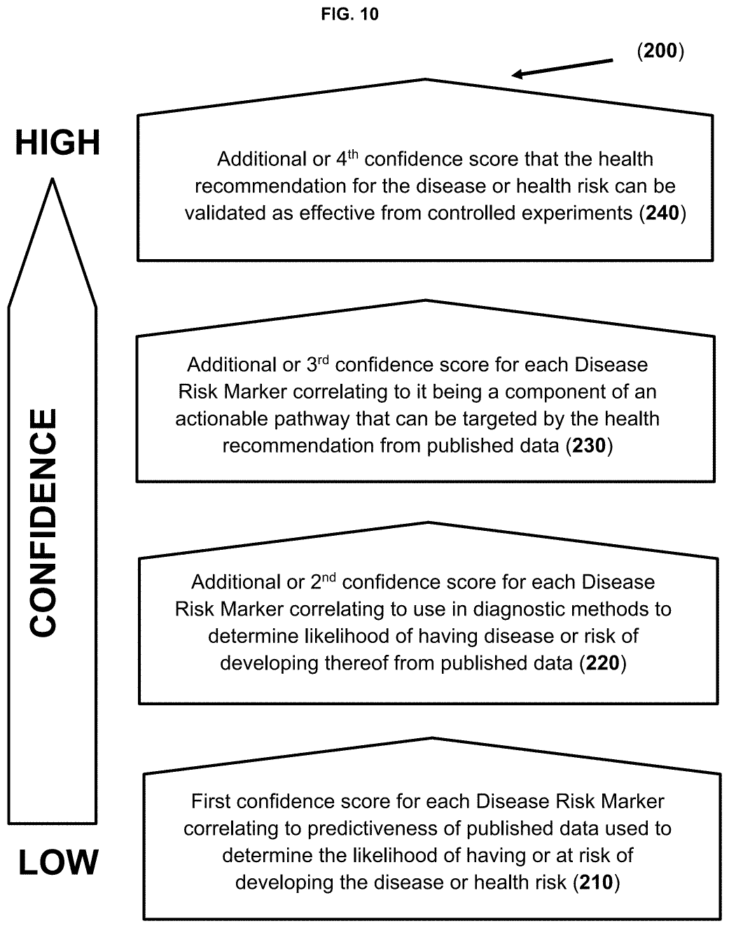

The method may employ a sequence of computer-readable instructions or computational steps that use multiple measures of confidence, which can then be stacked to form a “confidence stack” or a “confidence pyramid” (200) (as shown in FIG. 10). By employing a confidence stack (200), the confidence level in the methodology is increased. Basically, the confidence score outlined herein above related to the strength of predictiveness of the published data used to determine the likelihood of having or at risk of developing the disease or health risk can comprise the first confidence score (210) that is stacked. Then additional confidence scores relating to other measures of the Disease Risk Markers can be calculated and stacked accordingly.

In another aspect of the present disclosure, the method further comprises determining whether each of the Disease Risk Marker is conventionally used in diagnostic methods to determine the likelihood of having or at risk of developing the disease or health risk. The predictive equation is determined based on the binary characteristic of whether a specific Disease Risk Marker is used in traditional or conventional medical practices as diagnostic criteria. For example, fasting blood glucose levels are routinely used in clinical practice to diagnose type 2 diabetes, and this characteristic is included as a weighting factor in the predictive equation. This binary score or confidence score may also be referred to as a “clinical/diagnostic evidence score”.

The determination involves multivariate regression analysis of published data of the human subjects that have the disease or health risk. The multivariate regression analysis comprises calculating an additional confidence score of the published data, wherein the additional confidence score relates to a measure of confidence of the use of each of the Disease Risk Marker in diagnostic methods to determine the likelihood of having or at risk of developing the disease or health risk. A weighted confidence score is then calculated of the published data based on inputs from all of the confidence scores. With continued reference to FIG. 10, the additional confidence score or second confidence score (220) relating to the measure of confidence of the use of each of the Disease Risk Marker in diagnostic methods is stacked with the first confidence score (210) to calculate the weighted confidence score.

In another aspect of the disclosure, the method further comprises determining whether each of the Disease Risk Marker is a component of an actionable pathway that can be targeted by a health recommendation (e.g., specific nutritional, exercise and/or supplemental lifestyle action). As used herein, the expression “actionable pathway” refers to the biomarker that can be targeted directly or indirectly to improve the influence of the activity or expression of other proteins in the pathway involved with the disease or health risk. The predictive equation is determined based on the binary characteristic of whether a specific Disease Risk Marker associated with a specific health recommendation is a component of an actionable pathway that can be targeted by the health recommendation. This binary score or confidence score may also be referred to as an “actionability evidence score”.

This determination involves multivariate regression analysis of published data of the human subjects that have the disease or health risk. The multivariate regression analysis comprises calculating an additional confidence score of the published data, wherein the additional confidence score relates to a measure of confidence that each of the Disease Risk Marker is the component of the actionable pathway that can be targeted by the health recommendation. A weighted confidence score is then calculated of the published data based on inputs from all of the confidence scores. With reference to FIG. 10, the additional confidence score or third confidence score (230) relating to the measure of confidence that each of the Disease Risk Marker is the component of the actionable pathway that can be targeted by the health recommendation is stacked with the first confidence score (210) and/or the second confidence score (220) to calculate the weight confidence score.

In another aspect of the present disclosure, the method further comprises determining whether a health recommendation for the disease or health risk can be validated in respect of efficacy. In such embodiment, the predictive equation is determined based on multivariate regression analysis of controlled experiments of human subjects that have the disease or health risk and exposed to the health recommendations. The multivariate regression analysis comprises calculating an additional confidence score of each of the controlled experiment, wherein the additional confidence score relates to a measure of confidence that the health recommendation for the disease or health risk can be validated as effective. This confidence score may also be referred to as an “internal validation evidence score”.

A weighted confidence score is then calculated from the published data based on inputs from all of the confidence scores. With reference to FIG. 10, the additional confidence score or fourth confidence score (240) relating to the measure of confidence that the health recommendation for the disease or health risk can be validated as effective is stacked with the first confidence score (210) and/or the second confidence score (220) and/or the third confidence score (230) to calculate the weight confidence score.

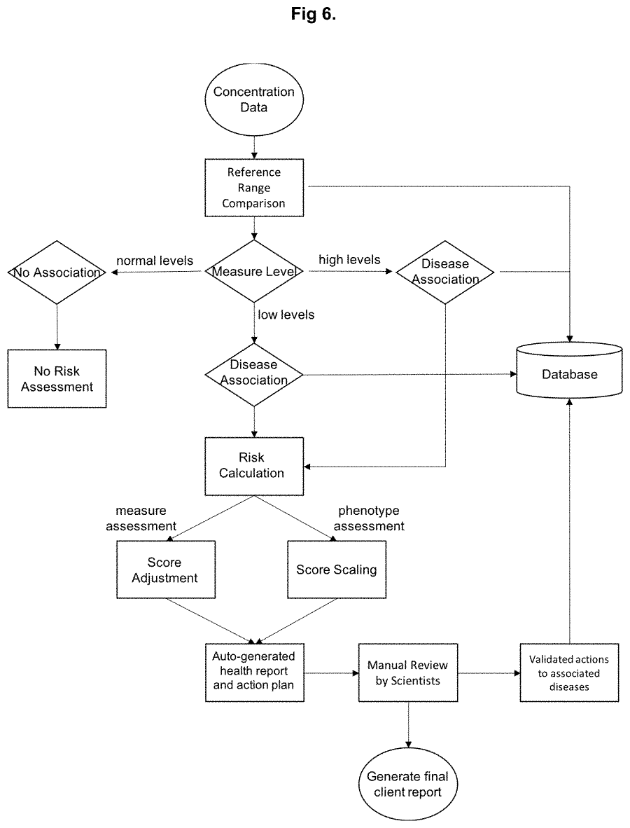

Methods, such as multivariate analysis of variance, i.e., multivariate regression analysis, can be carried out by those of skill the art. Multivariate regression analysis techniques consider multiple parameters separately so that the effect of each parameter may be estimated. A brief description of the process is shown in FIG. 6. The inputs for the Risk Calculation, using multivariate regression analysis, relies on various inputs including Disease Risk Markers from both scientific literature and an individual's sample measurements. Alternatively, the inputs for the Risk Calculation can be derived from various inputs from Disease Risk Markers from controlled experiments. The multivariate regression model may be adjusted by those of skill in the art based on score adjustment and scaling parameters (for example, if the individual indicated they have/had the disease in their self-reported phenotype form). In one embodiment, the output of the multivariate regression models is evaluated for goodness of fit before a final health status report is generated for the client.

Of course, one skilled in the art will recognize that embodiments other than those described herein may be utilized to prepare predictive equations and/or to increase the accuracy of the predictions of the predictive equations. The standard issues that affect prediction from using multivariate regression analysis are present, such as over-fitting of the model. Therefore, in one embodiment, an assessment of the goodness of fit and model diagnostics are carried out for each regression for each disease at a time. Furthermore, any new Disease Risk Markers to disease associations (i.e., new predictive variables) that need to be introduced, such as those based on new research, will result in changes to the predictive equations that can increase the accuracy of these equations.

Returning to the method (10) as depicted in FIG. 1, the method (10) may optionally comprise counseling the individual with respect to health recommendation for improving the health status, wherein the health recommendation is based on the magnitude of the gap (block 14B). The “magnitude of the gap” is calculated by the platform and refers to the magnitude of difference between calculated scores from the individual's sample Disease Risk Markers and a score calculated from published Disease Risk Markers. The “magnitude of the gap”, i.e., the mathematical difference of a disease score from published Disease Risk Markers and disease score from an individual's sample Disease Risk Markers indicates the health status of the subject. In one embodiment, the method comprises recommending dietary changes, nutritional supplements or both suitable for improving the health status of the individual.

With continued reference to FIG. 1, the method (10) further comprises identifying and verifying health recommendations that improve health status of the individual by confidence score increase (block 15). Basically, as individuals receive their health reports and follow the health recommendations, monitoring is undertaken to confirm which health recommendations improved the disease or health risk in the individual. Health recommendations that have led to improvements in the disease or health risk are then flagged. The sequence of operating steps are updated to incorporate the health recommendations linked to specific Disease Risk Markers having the disease or health risk that were improved.

In another aspect, the present disclosure is directed to a method of determining, based on a set of Disease Risk Markers corresponding to a disease or health risk, a magnitude of a gap between sampled Disease Risk Markers and published Disease Risk Markers of a human subject to determine a health status. The method comprises analyzing at least 25, preferably at least 20, preferably at least 15, preferably at least 10 or preferably at least 5 sampled Disease Risk Markers of the human subject to determine measurement data indicative of a disease or a health risk or a risk of developing thereof of a human subject, wherein the at least 25, preferably at least 20, preferably at least 15, preferably at least 10 or preferably at least 5 measurement data corresponds to the disease or health risk. In certain embodiments, the method comprises analyzing at least 300, 275, 250, 225, 200, 175, 150, 125, 100, 95, 90, 85, 80, 75, 70, 65, 60, 55, 50, 45, 40, 35, 30, 35, 20, 15, 10, or 5 sampled Disease Risk Markers of the human subject to determine measurement data. In certain embodiments, the measurement data corresponds to at least 100, 90, 80, 70, 60, 50, 40, 30, 20, 15, 10, 5, 2 or 1 of the disease or health risk.

The method further comprises determining the absence or presence of polymorphisms in the sampled Disease Risk Markers or levels of the sampled Disease Risk Markers from the measurement data from the subject, and calculating, by a computer device and based on the least 25, preferably at least 20, preferably at least 15, preferably at least 10 or preferably at least 5 measurement data, a magnitude of a gap between the sample Disease Risk Markers and corresponding published Disease Risk Markers, wherein each Disease Risk Marker is correlated with affecting one or more of the disease or health risk, wherein the magnitude of the gap indicates the health status of the subject.

In one embodiment, the disease or health risk or risk of developing thereof is determined based on applying a predictive equation, wherein the predictive equation corresponds to the disease or health risk or the risk of developing thereof and is determined by multivariate regression analysis of published data of human subjects that have the disease or health risk.

In another aspect, the present disclosure is directed to a method of determining thresholds for different biological pathways, which the inventors have termed “Body Functions” (also referred to as “organ health”), associated with the development of the disease or health risk. “Body Functions” as used herein generally relate to specific physiological processes and may involve multiple organ systems that influence an individual's overall health status. Suitable non-limiting examples of Body Functions may include: coagulation, lipid metabolism, inflammation, immune response, ageing, nutrition and/or dietary health, cognitive health, kidney health, liver health, oxidative stress, disease protection and insulin resistance.

Coagulation, also known as blood clotting, is the process by which blood changes from a liquid to a gel, forming a blood clot. It may result in hemostasis, which is the cessation of blood loss from a damaged vessel, followed by repair. Coagulation involves a number of biomarkers (i.e., molecular mediators) and follows through processes, including, but not limited to activation, adhesion and aggregation of platelets along with deposition and maturation of fibrin clot that may be useful for the evaluation of Body Functions.

Lipid metabolism includes measures that may be involved in both the processes of synthesizing fats (i.e., lipogenesis) and the breakdown and storage of these fats for energy.

Inflammation includes measures that are involved in the complex biological response of the body's tissues to harmful stimuli, such as pathogens, damaged cells or other irritants. Inflammation pathway is a protective response involving immune cells, blood vessels and many biomarkers (e.g., molecular meditators) to eliminate the initial cause of the cell injury and initiate tissue regeneration and repair. Inflammation is the body's natural response to infection, illness or injury. The discussion below is divided into four categories: Acute Inflammation, Chronic Inflammation, Systemic Inflammation, and Vascular Inflammation, to provide a more detailed illustration of the inflammatory processes occurring in the body.

In acute inflammation, there may be symptoms such as swelling, redness, heat, and pain. It is an important part of healing and generally lasts for less than 2 weeks. However, when the body experiences stress over a longer time span, the inflammation may become chronic. Toxins, excess fat, allergens, gut microbiome dysfunction, overtraining, and many other factors contribute to chronic inflammation. When the body has an inflammatory response to a stimulus, this is known as systemic inflammation. Systemic inflammation can be chronic or acute. Inflammation can also occur in the blood vessels. This process is called vascular inflammation. It causes blood vessel damage, which produces specific signals. Choosing foods rich in omega-3 fatty acids, avoiding red meat and processed foods, and light-to-moderate exercise can lower inflammation.

Hormone regulation includes measures that are involved in the regulation, transport and/or regulating the effects of circulating active hormones in the body.

Immune health includes measures that are involved in how the immune system performs its function and regulation involved in the processes that are involved in immune system development, pathogen surveillance methods in the innate immune system, evolving immunity in the adaptive immune system, and regulation of both the inflammatory and anti-inflammatory mechanisms of the immune response. Dysfunction of these measures may lead to the development of immunodeficiency or autoimmunity.

Ageing represents the accumulation of physical, physiological and social changes that occur in an individual over time. Ageing may be caused by a number of mechanisms. For example, the accumulation of damage via DNA oxidation damage may cause biological systems to fail or decrease in the hydrochloric acid production with increased age. As a result, the individual loses or has impaired ability to digest proteins which are needed for normal cellular process, tissue repair and regeneration.

Nutrition and/or Dietary Health involves the interaction of nutrients and other substances in food in relation to the proper maintenance, growth, reproduction, and health status of an individual. For the purposes of the present disclosure, biomarkers involved in food breakdown, absorption, assimilation, biosynthesis, catabolism and excretion may be useful measures to analyze in order to assess Body Functions.

Oxidative stress is understood as an imbalance between the production of free radicals and the body's ability to counteract or detoxify their harmful effects through neutralization by antioxidants. Free radicals are oxygen containing molecules that contain one or more unpaired electrons, making it highly reactive with other molecules. Typically, free radicals chemically interact with cell components such as, for example, DNA, proteins, or lipid and steal their electrons in order to become stabilized, in turn, destabilizing the cell component molecules which may trigger large chain of free radical reactions. Biomarkers connected to oxidative stress may be useful to assess Body Functions.

Disease protection (i.e., disease prevention and organ protection) may have key protective roles in preventing the pathogenesis or exacerbation of disease. These measures may also be involved in protecting organ systems from damage and deterioration. Biomarkers connected to Disease protection may be useful to assess Body Functions.

Insulin resistance or sensitivity describes how the body reacts to the effects of insulin. An individual said to be insulin sensitive will require smaller amounts of insulin to lower blood glucose levels than an individual who has low sensitivity. Insulin resistance implies that the cells are not responding well to the hormone insulin. This causes higher insulin levels, higher blood sugar levels and may lead to type 2 diabetes and other health problems. Biomarkers connected to Insulin resistance or sensitivity may be useful to assess Body Functions.

Cognitive health includes measures encompasses reasoning, memory, attention and other intellectual functions, which the brain executes. While the brain makes up only 2% of total body weight, it uses more than 20% of the energy that is produced. Glucose and fat are the key energy sources for the brain. Amino acids help to transport these nutrients across the blood-brain barrier. Blood vessel health, inflammation, vitamins and minerals also contribute to cognitive health. As the brain uses more energy than any other organ, cognition ability tends to be sensitive to changes in these contributing markers. Regular exercise, a healthy diet, and intellectual and social stimulation contribute to maintenance of proper cognitive health.

Liver health includes measures that are associated with liver function and maintenance of the biological systems that are associated with proper liver function. The liver is a critical organ that performs over 500 functions vital for life. It is the primary detoxification organ, and also plays a role in aiding digestion, making energy, and balancing hormones. It processes everything that is consumed, including all medications, supplements, and chemical exposures. Most proteins, including those involved in wound healing and immune processes, are made in the liver as well. The liver is resilient and will continue to function, even if two-thirds of it has been damaged. Despite this, blood markers can help to identify the health of the liver. Eating a healthy diet, reducing or avoiding alcohol consumption, and exercising caution with over-the-counter drugs and supplements contribute to maintenance of proper liver function.

Kidney health includes measures that are associated with kidney function and maintenance of proper kidney function. The kidneys are two fist-sized organs underneath the rib cage. They regulate blood pressure and filter wastes and toxins from the blood. They also activate Vitamin D, build red blood cells, and keep electrolytes in balance. The kidneys play an important role in overall health, but the early symptoms of poor kidney health are not obvious. Markers in the blood offer signs of how well the kidneys are functioning. Eating a healthy diet and maintaining a healthy weight can help maintain kidney functionality.

It will be noted that the method of assessing the Body Functions of an individual will work in a substantially similar manner as the method for assessing health status. In particular, the method of assessing the Body Functions involves determining thresholds of the different biological pathways in subjects having the disease or health risk and determining confidence score for these correlations.

Specifically, the present disclosure is directed to a method for assessing Body Functions of an individual. The method comprises providing a biological sample obtained from the individual; measuring at least 25, preferably at least 20, preferably at least 15, preferably at least 10 or preferably at least 5 Disease Risk Markers in the biological sample selected from the group consisting of Genomic Markers, Proteomic Markers, Metabolomic Markers, Exposomic Markers and a combination thereof to provide measurement data from the sample in relation to the human subject; and determining a predicted health status corresponding to the Body Functions, by applying a predictive equation corresponding to the measurement data to the Body Functions. In certain embodiments, the method comprises measuring at least 300, 275, 250, 225, 200, 175, 150, 125, 100, 95, 90, 85, 80, 75, 70, 65, 60, 55, 50, 45, 40, 35, 30, 35, 20, 15, 10, or 5 sampled Disease Risk Markers in biological sample to provide measurement data from the sample in relation to the human subject.

Optionally, the method described herein comprises measuring at least two, at least three or all four Disease Risk Markers selected from the group consisting of Genomic Markers, Proteomic Markers, Metabolomic Markers and Exposomic Markers. Thus, in one aspect, the method of the disclosure provides information regarding an individual's Body Functions or risk of developing disease or health risk associated with the Body Functions based on four different biologic biomarkers, which allows a more comprehensive and accurate evaluation of an individual's Body Functions.

In one embodiment, the predictive equation corresponds to the Body Functions and is determined by multivariate regression analysis of published data of human subjects that have the disease or health risk. The multivariate regression analysis comprises calculating a confidence score of each of the published data of the human subjects and the published data comprises a plurality of measurements corresponding to each human subject to the Body Functions. The plurality of measurements are associated with biological pathways involving complex networks of Proteomic Markers, Metabolomic Markers, and Exposomic Markers, called Body Functions, and determined from published Disease Risk Markers of each human subject in the published data. The predicted health status is representative of the human subject having the disease or health risk or risk of developing thereof.

Preferably, in the method of the present disclosure, the step of determining Body Functions comprises comparing the sampled Disease Risk Markers to the published Disease Risk Markers associated with the disease or health risk; and determining a magnitude of a gap between the sampled Disease Risk Markers and the published Disease Risk Markers.

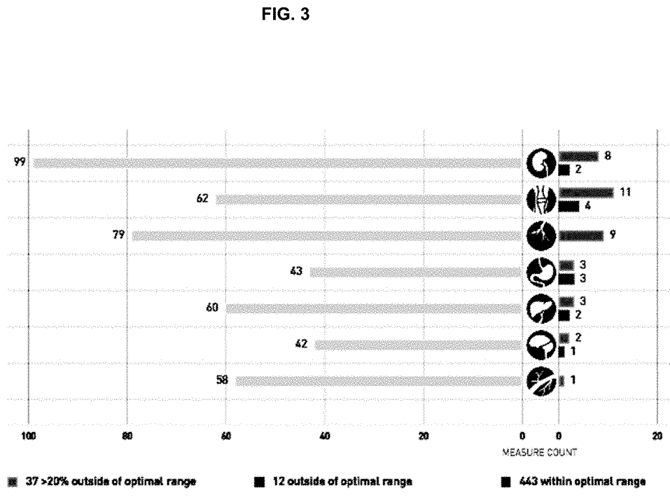

FIG. 3 provides an exemplary Body Functions assessment of an individual across 10 measures. For example, the inventors identified 10 biofunctions that are associated with early disease pathogenesis and using similar techniques to predict disease risks from biomarker levels, the inventors were able to score biofunction risks from the biomarker levels. This was accomplished by categorizing each of the measured biomarkers into 10 biofunction bins (as shown in FIG. 3). The biomarkers that are outside the normal ranges are indicated by lighter shades of gray, depending on the magnitude of the level of deviation from normal ranges. The more biofunctions that fall into the lighter gray ranges, the more association there is to the specific biofunction, and a specific score was assigned. As part of the Body Functions assessment, the individual may optionally receive personalized counseling for a plan containing actionable measures (e.g., dietary and supplement recommendations) in order to decrease the health risks and normalize the biomarkers outside of the optimal ranges. Ideally, the action plan would be based on the published research data linking nutrient intake and dietary patterns to metabolic and proteomic marker levels as well as genetic polymorphisms.

In one aspect of the disclosure, as previously discussed above, the confidence score is a weight confidence score, which is made up of a stacked or layered combination of more than one confidence score calculated from various measures including: (i) publication evidence score, (ii) clinical/diagnostic evidence score, (iii) actionability evidence score, and/or (iv) internal validation evidence score. The weight confidence score (i.e., stacked confidence score) is visualized as a pyramid or layer visual graph (as shown in FIG. 10) in the auto-generated final client health report for the strength of evidence for each Disease Risk Marker.

### Systems for Assessing Health Status

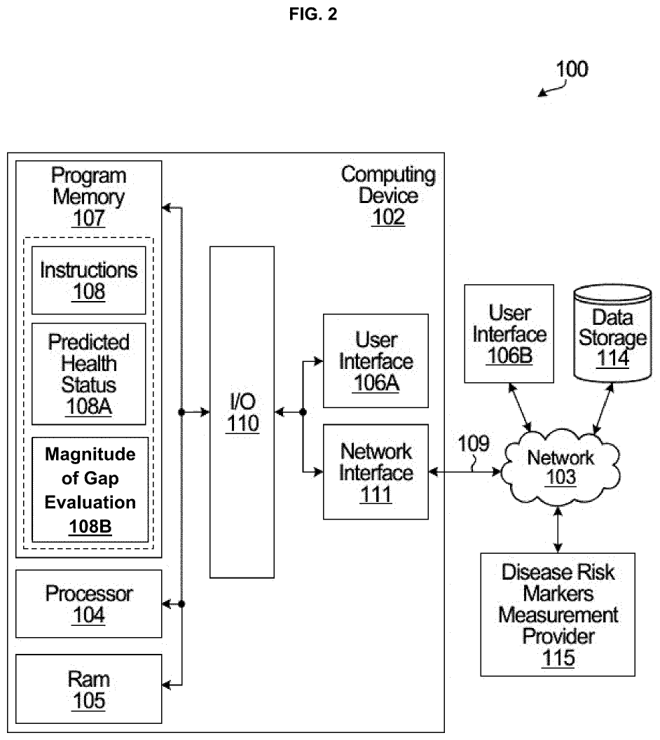

While the present disclosure is not dependent on a particular system, systems for use in the context of the method of the present disclosure, in one embodiment, have one or more of the features described herein. Turning now to FIG. 2, there is illustrated an embodiment of a system (100) for performing the method as described herein, specifically a method for assessing the health status of an individual or a method for assessing Body Functions of an individual. The system (100) is a platform that integrates multi-omics measurements to assess and/or predict an individual's risk of disease or health risk. The system (100) may further allow monitoring and comparison across multiple time points and disease clusters to support more effective and/or comprehensive medical care. In one embodiment, the system (100) may perform at least a portion of the method of assessing the health status of an individual or assessing the Body Functions of an individual.

In the illustrated embodiment as shown in FIG. 2, the system (100) may include a computing device (102) which may be, for example, a computer, a hand held device, a plurality of networked computing devices, a plurality of cloud computing devices, etc. Accordingly, for ease of discussion only and not for limitation purposes, the computing device (102,) is referred to herein using the singular tense, although in some embodiments the computing device (102) may include more than one physical device. In an embodiment, the computing device (102) may be physically located with the individual, and may be remotely accessible by the healthcare practitioner. In an embodiment, the computing device (102) may be a web server that is remotely located from the individual, but is communicatively accessible to the healthcare practitioner with a web server via a network (e.g., internet) (103), a website, a portal or the like.

The computing device (102) may comprise at least one processor (e.g., a controller, a microcontroller or a microprocessor) (104), a random-access memory (RAM) (105), an interface (106), a program memory (107) and an input/output (I/O) circuit (110), each of which may be interconnected via an address/data bus. In an embodiment, the interface (106) may comprise a display and input devices including a keyboard and/or a mouse. The program memory (107) may comprise at least one tangible, non-transitory computer readable storage medium or devices, in an embodiment. The at least one tangible, non-transitory computer readable storage medium or devices may be configured to store computer-executable instructions (108) that, when executed by the at least one processor (104), cause the computing device (102) to implement the method (10) of assessing the health status of an individual or another method of assessing Body Functions of an individual.

The instructions (108) may include a first portion (108A) for obtaining, via a Disease Risk Markers measurement provider (115), an indication of the presence, absence or level of Disease Risk Markers in a biological sample from the individual; and determine, based on the indication of the presence, absence or level of the sampled Disease Risk Markers, a predicted health status corresponding to a disease or health risk or a risk of developing thereof. For ease of discussion, the first portion instructions (108A) are referred to herein as a “predicted health status” (108A), and in an embodiment, the predicted health status (108A) performs block 14 of the method (10) as shown in FIG. 1.

Additionally or alternatively, the instructions (108) may include a second portion (108B) for comparing the sampled Disease Risk Markers to the published Disease Risk Markers associated with the disease or health risk; and determining a magnitude of a gap between the sampled Disease Risk Markers and the published Disease Risk Markers. For ease of discussion, the second portion instructions (108B) are referred to herein as a “magnitude of the gap evaluator” (108B) and in an embodiment, the magnitude of the gap evaluator (108B) may determine a magnitude of a gap between the sampled Disease Risk Markers and the published Disease Risk Markers, and may cause an indication of the gap magnitude to be presented at a user interface (106) or at a remote user interface.

Additionally or alternatively, one or more other sets of computer-executable instructions (108) may be executable by the processor (104). In an embodiment, the one or more other sets of computer executable instructions (108) may be executable by the processor (104) for causing the system (100) to: generate the health status report and to suggest health recommendations such as, for example, identify dietary changes, nutritional supplements or both suitable for improving the health status of the individual; and present the identity of the dietary changes, the nutritional supplements or both at a user interface (106A).

In another embodiment, the one or more other sets of computer executable instructions (108) may be executable by the processor (104) for causing the system (100) to: determine, based on the sampled Disease Risk Markers, a respective current health status corresponding to each disease or health risk included in the group of the diseases or the health risk; determine a respective magnitude of a respective gap between the respective predicted health status and the respective current health status for each disease or health risk included in the group of the diseases or health risk; identify a specific disease or health risk associated with the determined gap magnitudes; and identify dietary changes, nutritional supplements or both suitable for improving the specific disease or health risk.

In yet another embodiment, the one or more other sets of computer executable instructions (108) may be executable by the processor (104) for causing the system (100) to: determine a subsequent health status of the individual from analysis of subsequent sampled Disease Risk Markers of the individual at a later time point; and determine a subsequent magnitude of a gap between the predicted health status and the subsequent health status of the individual.

The system (100) may be configured or adapted to access or receive data from one or more data storage devices (114). For example, the instructions (108) may be executable by the processor (104) to access the one or more data storage devices (114) or to receive data stored on the data storage devices (114). Additionally or alternatively, one or more other sets of computer executable instructions (108) may be executable by the processor (104) to access or receive data from the one or more data storage devices (114).

The one or more data storage devices (114) may comprise, for example, one or more memory devices, a data bank, cloud data storage, or one or more other suitable data storage devices. In the embodiment illustrated in FIG. 2, the computing device (102) is shown as being configured to access or receive information from the one or more data storage device (114) via a network or communications interface (103) that is coupled to a link (109) in communication connection with the one or more data storage devices (114). The link (109) in FIG. 2 is depicted as a link to one or more private or public networks (103) (e.g., the one or more data storage devices (114) are remotely located from the computing device (102)), although is not required. The link (109) may include a wired link and/or a wireless link, or may utilize any suitable communications technology.

In an embodiment (not shown), at least one of the one or more data storage devices (114) is included in the computing device (102), and the processor (104) of the computing device (102) (or the instructions (108) being executed by the processor (104)) accesses the one or more data storage devices (114) via a link comprising a read or write command, function, primitive, application programming interface, plug-in, operation, or instruction, or similar.

The one or more data storage devices (114) may include on a physical device, or the one or more data storage devices (114) may include more than one physical device. The one or more data storage devices (114), though, may logically appear as a single data storage device irrespective of the number of physical devices included therein. Accordingly, for ease of discussion only and not for limitation purposes, the data storage device (114) is referred to herein using the singular tense.

The data storage device (114) may be configured or adapted to store data related to the system (100). For example, the data storage device (114) may be configured or adapted to store one or more predictive equations, each of which may correspond to published data on the Disease Risk Markers (e.g., Genomic Markers, Proteomic Markers, Metabolic Markers, Exposomic Markers) and their correlation to diseases or health risks or a risk of developing thereof In an embodiment, the predictive equations include at least the equations discussed above with respect to FIG. 1.

In an embodiment, the “predicted health status” (108A) is configured or adapted to determine the predicted health status (block 14) of the individual based on one or more of the predictive equations. The predicted health status (108A) may query the data storage device (110) for the one or more predictive equations as needed, and/or the one or more predictive equations may be delivered to or downloaded to the computing device (102) a priori. The predictive equation is determined by multivariate regression analysis of published data of human subjects that have the disease or health risk. The multivariate regression analysis comprises calculating a first confidence score of each of the published data of the human subjects. The first confidence score relates to a measure of confidence on the strength of predictiveness of the published data used to determine the likelihood of having or at risk of developing the disease or health risk. The published data comprises a plurality of measurements corresponding to each individual that has the disease or health risk. The plurality of measurements is associated with the disease or health risk and determined from published Disease Risk Markers of each human subject in the published data. The health status is representative of the individual having the disease or health risk or risk of developing thereof.

With continued reference to FIG. 2, a Disease Risk Markers measurement provider (115) may perform an analysis on a biological sample obtained from the individual to determine the plurality of measurements of the Disease Risk Markers corresponding to the diseases or health risks. In an embodiment, the Disease Risk Markers measurement provider (115) is configured to both obtain the samples and perform the analysis. For example, Disease Risk Markers measurement provider (115) may be a clinic or laboratory that obtains the biological samples from the individual and then analyzes them for an indication of the presence, absence or level of Disease Risk Markers. The Disease Risk Markers measurement provider (115) is configured to cause the plurality of sampled measurement data from the individual to be delivered to the computing device (102).

In an embodiment, the Disease Risk Markers measurement provider (115) may be remotely located from the computing device (102) and may cause the sampled measurements to be transmitted to the computing device (102) using the network (103) and the network interface (111) so that the predicted health status (108A) may determine a predicted health status (block 14). In an embodiment, in addition to determining the plurality of sampled measurements corresponding to the Disease Risk Markers correlated to the diseases or health risks, the Disease Risk Markers measurement provider (115) may also cause the transmission to the magnitude of the gap evaluator (108B) of the computing device (102) to determine a magnitude of a gap between the sampled Disease Risk Markers and the published Disease Risk Markers.

Turning again to the computing device (102) in FIG. 2, while the predicted health status (108A) is shown as a single block, it will be appreciated that the predicted health status (108A) may include a number of different programs, modules, routines, and sub-routines that may collectively cause the computing device (102) to implement the predicted health status (108A). In an embodiment, the predicted health status (108A) may be executable by the processor (104) to cause the computing device (102) to determine a presence or absence of one or more polymorphisms in the Genomic Markers. For example, the indication of the presence or absence of the one or more polymorphisms may have been determined from an analysis of nucleic acid from a biological sample from the individual, as described elsewhere herein. Further, the presence of absence of the one or more polymorphisms may be associated with diseases or health risks, and the associated diseases or health risks are indicative of the predicted health status of the individual.

In another embodiment, the predicted health status (108A) may be executable by the processor (104) to cause the computing device (102) to determine levels of one or more of the Disease Risk Markers (e.g., Proteomic Markers, the Metabolic Markers, the Exposomic Markers) in the biological sample. For example, the indication of the levels of the one or more biomarkers may have been determined from an analysis of biological samples (e.g., bodily fluids) from the individual, as described elsewhere herein. Further, the levels of the one or more biomarkers may be associated with diseases or health risks, and the associated disease or health risks are indicative of the predicted health status of the individual.

In one embodiment, the predicted health status (108A) may be further executable by the processor (104) to determine, for each polymorphism whose presence or absence was determined, a respective predictive health status to each disease or health risk. The predicted health status (108A) may be further executable by the processor (104) to determine, based on the biological sample, a respective current health status corresponding to each disease or health risk, and to determine a respective magnitude of a respective gap between the respective predicted health status and the respective current health status for each disease or health risk. In an embodiment, the predicted health status (108A) may be further executable by the processor (104) to cause the assessed health status to be presented at a user interface (106).

Similarly, while the magnitude of the gap evaluator (108B) is shown as a single block, it will be appreciated that the other instructions for evaluating a magnitude of the gap between the sampled Disease Risk Markers and the published Disease Risk Markers may include a number of different programs, modules, routines, and sub-routines that may collectively cause the computing device (102) to implement the other instructions for evaluating the magnitude of the gap evaluator (108B). In an embodiment, the magnitude of the gap evaluator (108B) may be executable by the processor (104) to cause the computing device (102) to receive first data that includes at least one indication of the presence or absence of at least one polymorphism or levels of the biomarkers, in a biological sample from the individual, indicative of a respective current health status of the individual, as described elsewhere herein. The magnitude of the gap evaluator (108B) may be further executable by the processor (104) to cause the computing device (102) to determine a value (i.e., magnitude of the gap) indicative of the respective current health status of the individual, where the respective current health status is determined based on the first data and on a correlation of the biomarkers to diseases or health risks in published research data.

Additionally, the magnitude of the gap evaluator (108B) may be executable by the processor (104) to cause the computing device (102) to receive second data that includes at least one indication of the presence or absence of at least one polymorphism or levels of the biomarkers, in a biological sample from the individual, indicative of a subsequent health status of the individual, as described elsewhere herein. The magnitude of the gap evaluator (108B) may be further executable by the processor (104) to cause the computing device (102) to determine a subsequent value (i.e., subsequent magnitude of the gap) indicative of the respective gap between the predicted health status and the subsequent health status of the individual. The magnitude of the gap evaluator (108B) may be further executable by the processor (104) to cause the computing device (102) to cause an indication of the subsequent magnitude of the gap be presented at a user interface (106), such as the user interface (106A) and/or the user interface (106B).

It should be appreciated that, although only one processor (104) is shown, the computing device (102) may include multiple processors (104). Additionally, although the I/O circuit (110) is shown as a single block, it should be appreciated that the I/O circuit (110) may include a number of different types of I/O circuits. Similarly, the memory of the computing device (102) may include multiple RAMs (105) and multiple program memories (107). Further, while the instructions (108) are shown in FIG. 2 as being stored in the program memory (107), the instructions (108) may additionally or alternatively, be stored in the RAM (105) or other local memory (not shown).

The RAM(s) (105) and program memories (107) may be implemented as semiconductor memories, magnetically readable memories, chemically or biologically readable memories, and/or optically readable memories, or may utilize any suitable memory technology. The computing device (102) may also be operatively connected to the network (103) via the link (109) and the I/O circuit (110). The network (103) may be a proprietary network, a secure public internet, a virtual private network or some other type of network, such as dedicated access lines, plain ordinary telephone lines, satellite links, combinations of these, etc. Where the network (103) comprises the internet, data communications may take place over the network (103) via an internet communication protocol, for example.

Additionally, the user interface (106) may be integral to the computing device (102) (e.g., the user interface (106A)), and/or the user interface may not be integral with the computing device (102) (e.g., the user interface (106B)). For example, the user interface (106) may be a remote user interface (106B) at a remote computing device, such as a web page or a client application. In any event, the user interface (106) may effectively be a communication interface between the computing device (102) and a user.

Additionally, to handle multiple vendor uploads of raw -omics mass spectrometry data into the system (100), a data processing system has been developed to handle the raw data. As part of the data processing system, it reads the data and generates health reports. Specifically, the data processing system initially reads entire raw files that comes in at once and saves the raw laboratory results to a database. It then processes the saved data in terms of setting the final ‘reportable’ concentrations, matching reference ranges, and assigning individual biomarker levels. Finally, a health data report is generated by assessing biomarkers associated to various health and body function risks. The data processing system that was developed is automated and able to handle large data sets in a timely manner by using a multi-level queueing system to handle individual samples with detailed tracking of where data is in the processing pipeline.

With this data processing system, once a vendor data file is received, each sample identifier may be placed in a high priority queue that manages jobs for saving data. This allows for the receipt of any amount of data files with many samples included, without overwhelming the system (100). With this setup, it is still possible to run multiple jobs at the same time, but limiting these according to memory and server needs, and with the ability to track each job status. This approach according to such embodiment can also save specific errors and send automated emails when these occur. Once completed, each sample moves on to the next data process individually. Each process has a different queue with a different priority setting. Once processing is done, only patients with complete data sets (e.g., metabolomics, proteomics, etc. profiles) are next queued to have a health data report generated. In one embodiment, a sample may progress from one process to the next regardless of whether or not each of the jobs in a ‘job batch’ are complete or successful. Identifiers become re-grouped with each type of process to speed up completion of reports. This process also enables individual components to be re-run for a given sample without having to reload an entire data batch. If errors are detected in the raw data (except for ones rejecting the data entirely) or the pipeline, successful entries are not held back.

### EXAMPLES

The following examples describe some exemplary modes of practicing certain methods that are described herein. It should be understood that these examples are for illustrative purposes only and are not meant to limit the scope of the systems and methods described herein.

### Example 1

This example demonstrates the significant relationship between the biomarkers, the predicted health status, and the health benefits via health recommendations (i.e., Lifestyle Action Plan). In particular, the example presents the practice of the invention in a case-control study of an individual (i.e., Fred) to diagnosis his predicted health status and customizes a lifestyle action plan containing dietary, exercise, and supplemental recommendations, in order to decrease his health risks and normalize the biomarkers which are outside of the normal range. The diagnosis and lifestyle action plan are based on the most recently published scientific evidence linking nutrient intake and dietary patterns to metabolomics and proteomic marker levels as well as genetic polymorphisms.

### Initial Consultation

a. Biological samples are obtained from Fred. The obtained samples are analyzed for the presence, absence and/or levels of the biomarkers using the aforementioned analytical techniques (i.e., multiple reaction monitoring mass spectrometry (MRM-MS), high performance liquid chromatography (HLPC), and liquid chromatography-mass spectrometry (LC_MS)). These methods are used to quantify, for example, the levels of genomic, metabolomic, proteomic, and/or exposomic biomarkers present in the obtained samples. The measurements are recorded.

b. While the analytical assessment is in progress, Fred is also asked to complete a self-reporting phenotype form. The purpose of the form is to elicit information about a number of characteristics for Fred, including but not limited to, age, sex, height, weight, family disease history, individual disease history and symptoms, diet diary, and/or physical activity. For example, Fred self-reported that he is a Caucasian male in his early 50s with a history of diabetes and had been diagnosed with pre-diabetes in the past. Fred's previous diagnosis resulted in changes in his lifestyle, including increasing his workout routine, training for a marathon and joining a High Intensity Interval Training (HITT) program. Since these changes, Fred had lost some weight, which allowed him to achieve a normal BMI. His glucose levels were normal from his last doctor's visit; as a result, Fred believed that he had overcome his risk of diabetes. Fred wanted to learn more about his health status given his lifestyle changes and participated in this case study.

c. Fred's measured biomarker levels are compared to a database of Disease Risk Markers which have been previously correlated to diseases or health risks according to the present invention. Risk scores are calculated for each disease that are reported on and these risks are categorized and ranked from highest risk score to lowest risk score based on the ‘magnitude of the gap’ technique (as described previously herein). Put another way, the Disease Risk Markers from Fred's biological sample and the Disease Risk Markers from published scientific data are compared and used to predict risk thresholds (i.e., divided into high risk, moderate risk, or low risk) that will represent Fred's Health Status.

d. ROB scores are calculated (as described previously herein) and displayed to represent the confidence in the strength of the association between each of the Disease Risk Markers and each of the Disease Risk. Fred's Health Status is displayed as high, moderate, or low risks of various diseases (referred to as Health Risks). For example, an electronic display generates a graphical depiction of the calculated confidence scores in the strength of the association between each of the Disease Risk Markers and each of the Disease Risk. FIG. 3 shows a bar chart visually summarizing the exemplary Body Functions assessment across 7 measures identified by the Applicant as being associated with early disease pathogenesis for diabetes. Fred's Health Risk of pre-diabetes is scored in the high risk zone due to a Disease Score that is calculated from the number of Disease Risk Markers that have measured levels outside of the published normal biomarker measured levels and are associated with pre-diabetes and any score scaling or algorithm adjustments that are in place for pre-diabetes. FIG. 3 reveals that Fred's risk for diabetes is driven by impaired lipid metabolism and inflammation. Further, the results indicated that Fred has several genetic markers that put him at higher risk for developing diabetes. Fred's metabolomic and proteomic biomarkers also indicated that he was likely consuming a diet high in saturated fats such as meat, full-fat dairy and eggs while being low in foods such as fish, nuts, legumes and whole grains (under “nutrition”). This did not benefit Fred's health and was likely a driver of his risk for diabetes, his impaired lipid metabolism and high inflammation.

e. Fred's predicted Health Status represented by Biofunctions is also calculated and categorized into the Biofunctions categories. For example, Fred's Health Risk of Inflammation was scored in the high risk zone due to a the number of Disease Risk Markers that have measured levels outside of the published normal biomarker measured levels and are associated with Inflammation and any score scaling or operating step adjustments that are in place for Inflammation.

f. Fred was surprised by the data he received as he did not expect to still be at high risk for diabetes based on the previous changes noted above in b. In fact, Fred was alarmed that his lifestyle choices might be contributing to his health risk rather than being beneficial for him. Therefore, it appears that Fred still needed help to reduce the risk of developing diabetes.

### Lifestyle Action Plan

g. Applicant also established a database of nutritional, supplement, and/or exercise actions (also called Lifestyle Actions) that can influence the levels of Disease Risk Markers and Health Risks based on data from published research studies. Specific biomarkers are identified and their levels that are associated with the diseases and compare to the database of lifestyle actions. Using this, Applicant is able to match various foods categories, exercises categories, micronutrients and/or supplements to the Disease Risk Markers that are outside of the normal ranges.

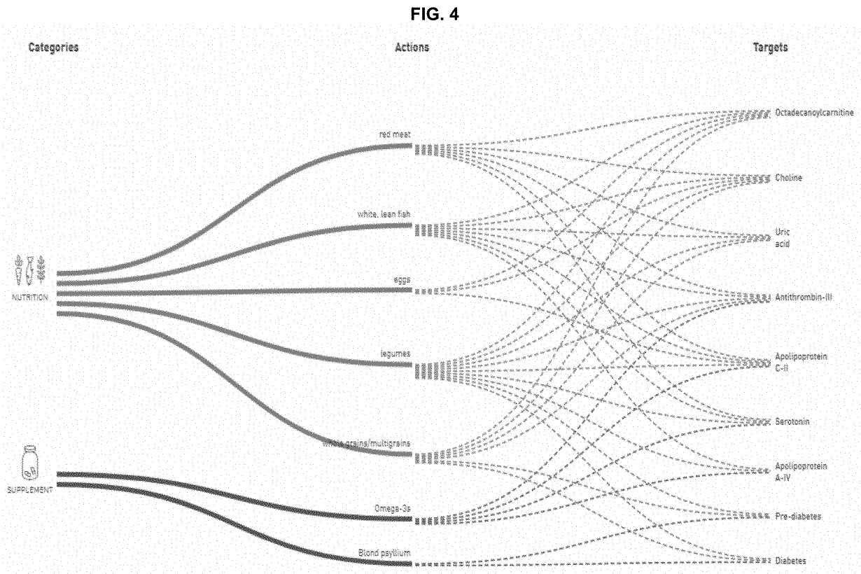

h. The goal is to generate a Lifestyle Action Plan for Fred, (as shown in FIG. 4) where certain of the lifestyle actions (e.g., nutrition, exercise, and/or supplements) can be undertaken by Fred to normalize his levels of identified and most critical Disease Risk Markers and Health Risks. For example, recommendations may change certain dietary, exercise, and/or supplement habits to decrease health risk and normal markers outside of the optimal range. Fred was provided with a personalized Lifestyle Action Plan with changes to his diet, especially the higher intakes of unsaturated fats and low intakes of animal fats and increases in fruits and vegetable consumption.

### Later Consultation

Fred followed the personalized Lifestyle Action Plan for four months. Fred continued his workout routine as before but otherwise made no further changes to his lifestyle. After the four-month period was over, biological samples were obtained from Fred and analyzed as described above.

Based on the test results, it appears that Fred was able to significantly improve his health, which was reflected in the decreased risk for diabetes. Any metabolic or proteomic indicators of high intakes of saturated fats normalized (data not shown). Fred's metabolic and proteomic profile shifted and reflected his changes in diets, especially the higher intakes of unsaturated fats and low intakes of animal fats.

### Example 2

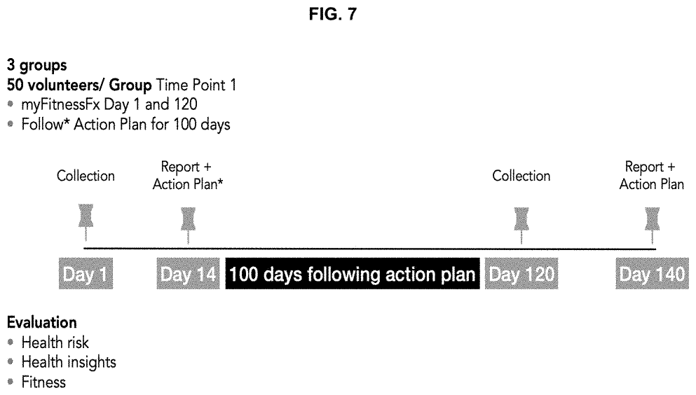

This example demonstrates the significant relationship between the biomarkers, the predicted health status, and the health benefits via health recommendations (i.e., Lifestyle Action Plan) at scale. In particular, the example presents a proof-of-concept study of multiple groups of study participants to diagnosis their predicted health statuses and customizing lifestyle action plans containing dietary, exercise, and supplemental recommendations, in order to decrease their health risks and normalize their biomarkers which are outside of the normal range. The diagnosis and lifestyle action plan are based on the most recently published scientific evidence linking nutrient intake and dietary patterns to metabolomics. The study design and timeline are represented in FIG. 7.

### Initial Consultation

a. Biological samples are obtained from the study participants. The obtained samples are analyzed for the presence, absence and/or levels of the biomarkers using the aforementioned analytical techniques (i.e., multiple reaction monitoring mass spectrometry (MRM-MS), high performance liquid chromatography (HLPC), and liquid chromatography-mass spectrometry (LC-MS). These methods are used to quantify, for example, the levels of genomic, metabolomic, proteomic, and/or exposomic biomarkers present in the obtained samples. The measurements are recorded.

b. While the analytical assessment is in process, the study participants were also asked to complete a self-reporting phenotype form. The purpose of the form is to elicit information about a number of characteristics for Fred, including but not limited to, age, sex, height, weight, family disease history, individual disease history and symptoms, diet diary, and/or physical activity. The study participants wanted to learn more about their health status given previous lifestyle changes before participating in this study.

c. The participants measured biomarker levels are compared to a database of Disease Risk Biomarkers which have been previously correlated to diseases or health risks or risks of developing thereof according to the present invention. Risk scores are calculated for each disease that are reported on and these risks are categorized and ranked from highest risk score to lowest risk score based on the ‘magnitude of the gap’ technique (as described previously herein). Put it another way, the Disease Risk Markers from participants biological samples and the Disease Risk Markers from published scientific data are compared and used to predict risk thresholds (i.e., divided into high risk, moderate risk, or low risk) that will represent the participants' Health Statuses.

d. An aggregate data analysis of metabolomics biomarker profiling of the study participants showed that around 20% of the population displayed moderate and high risks to their Health Status at the first timepoint for at least one of the nine analyzed diseases that are informed through metabolomics biomarker profiling, including Type 2 Diabetes and Alzheimer's disease (data not shown).

e. The participants' predicted Health Status represented by Biofunctions (or Body Functions) is also calculated and categorized into the Biofunctions categories. An aggregate analysis of the Health Status represented by Biofunctions (or Body Functions) revealed that, at the first timepoint, the majority of participants (68%) showed abnormal levels of metabolite biomarkers that represent the early indicators and causal factors of chronic diseases.

### Lifestyle Action Plan

f. Applicant also established a database of nutritional, supplement, and/or exercise actions (also called Lifestyle Actions) that can influence the levels of Disease Risk Markers and Health Risks based on data from published research studies. Specific biomarkers are identified and their levels that are associated with the diseases and compared to the database of lifestyle actions. Using this, Applicant is able to match various food categories, exercise categories, micronutrients and/or supplements to the Disease Risk Markers that are outside of the normal ranges.

g. The goal is to generate a Lifestyle Action Plan for each of the study participant where certain targeted lifestyle actions (e.g., nutrition, exercise, and/or supplements) can be undertaken by participants to normalize their levels of identified and most critical Disease Risk Markers and Health Risks. For example, recommendations may change certain dietary, exercise, and/or supplement habits to decrease health risk and normal markers outside of the optimal range.

h. The study participants were all provided with personalized Lifestyle Action Plans based on their Disease Risk Markers, Health Risks, and current personal lifestyle. After following their Action Plans for 100 days, the participants were profiled a second time, at the second timepoint, to determine the impact on their Disease Risks, Health Risks and Biofunctions/Body Functions.

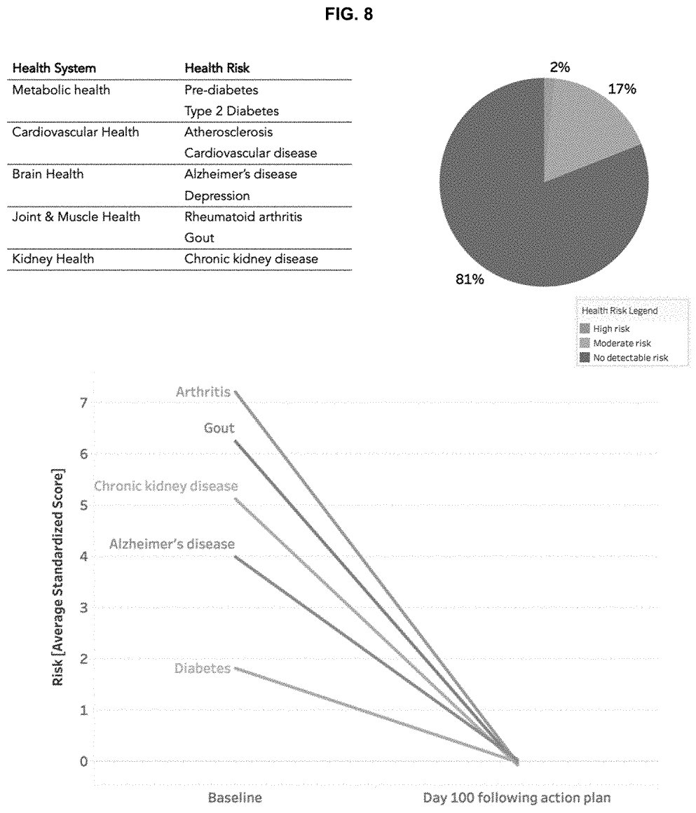

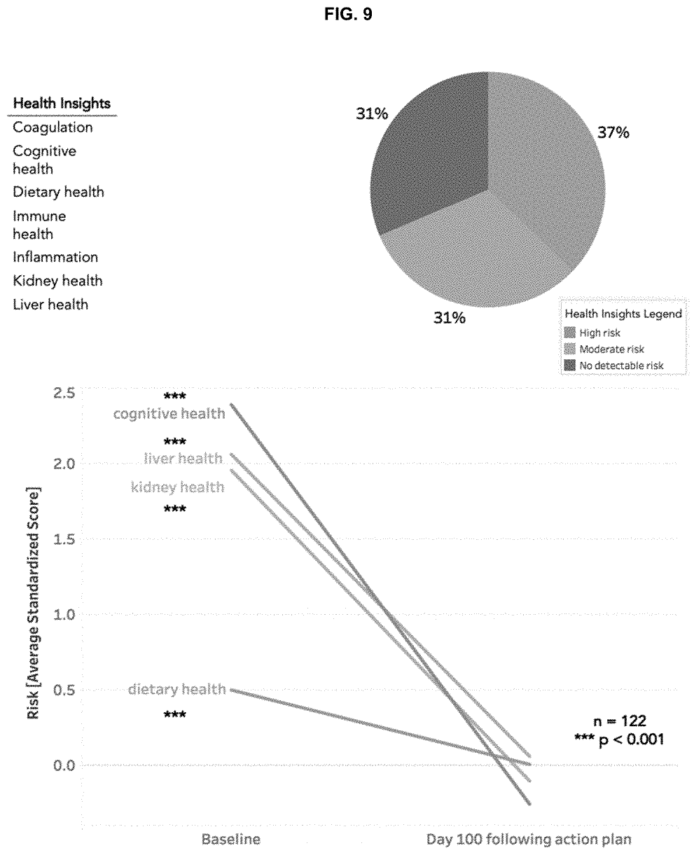

Aggregate analysis of participants Disease Risks and Health Risks showed a reduction of Disease Risks, including Type 2 Diabetes and Alzheimer's Disease, for example, at the second timepoint (see FIG. 8). There was also a significant reduction in abnormal metabolite biomarkers levels as indicated by reduction of Biofunctions/Body Functions scores (see FIG. 9).

Other examples of implementations will become apparent to the reader in view of the teachings of the present description and as such, will not be further described here.

Note that titles or subtitles may be used throughout the present disclosure for convenience of a reader, but in no way should these limit the scope of the invention. Moreover, certain theories may be proposed and disclosed herein; however, in no way should such theories, whether correct or incorrect, limit the scope of the invention so long as the invention is practiced according to the present disclosure without regard for any particular theory or scheme of action.

Elements of the methods and/or systems of the disclosure described in connection with the examples apply mutatis mutandis to other aspects of the disclosure. Therefore, it goes without saying that the methods and/or systems of the present disclosure encompasses any methods and/or systems comprising any of the steps and/or components cited herein, in any embodiment wherein each such step or component is independently present as defined herein. Many such methods and/or systems, other than what is specifically set out herein, can be encompassed by the scope of the invention.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”. The term “about” encompasses +/−5% deviation from a given value.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any disclosure disclosed or claimed herein or that it alone, or in any combination with any other reference or references, teaches, suggests or discloses any such disclosure. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the scope of the present disclosure. It is therefore intended to cover in the appended claims all such changes and modifications that are within the scope of this disclosure.

What is claimed is:

1. A method for assessing the health status of an individual, the method comprising:
providing a biological sample obtained from the individual;
measuring at least 25, preferably at least 20, preferably at least 15, preferably at least 10 or preferably at least 5 Disease Risk Markers in the biological sample selected from the group consisting of Genomic Markers, Proteomic Markers, Metabolomic Markers, Exposomic Markers and a combination thereof to provide measurement data from the sample in relation to the individual; and
determining a predicted health status corresponding to a disease or health risk or a risk of developing thereof, by applying a predictive equation corresponding to the disease or health risk or the risk of developing thereof to the measurement data, wherein the predictive equation is determined by a computer implemented multivariate regression analysis of published data of human subjects that have the disease or health risk,
wherein the computer-implemented multivariate regression analysis comprises outputting a confidence score of each of the published data of the human subjects and the published data comprises a plurality of measurements corresponding to each human subject that have the disease or health risk,
wherein the plurality of measurements correspond to each Disease Risk Marker associated with the disease or health risk and determined from published Disease Risk Markers of each human subject in the published data, and
wherein the predicted health status is representative of the individual having the disease or health risk or the risk of developing thereof.

2. The method according to claim 1, wherein the step of determining the predicted health status further comprises:
comparing the measured Disease Risk Markers to the published Disease Risk Markers associated with the disease or health risk; and
determining a magnitude of a gap between the measured Disease Risk Markers and the published Disease Risk Markers.

3. The method according to claim 2, further comprising providing a health recommendation, wherein the health recommendation is selected from the group consisting of: dietary changes, nutritional supplements, exercise actions or a combination thereof, suitable for improving the health status of the individual.

4. The method according to claim 3, wherein the recommendation is based on the magnitude of the gap.

5. The method according to claim 1, further comprising:
determining a respective predicted health status for each of the disease or health risk.

6. The method according to claim 5, further comprising:
determining, based on the sampled measurement data of the individual, a respective current health status corresponding to each of the disease or health risk; and
determining a respective magnitude of a respective gap between the respective predicted health status and the respective current health status for each of the disease or health risk.

7. The method according to claim 6, further comprising:
determining a subsequent health status of the individual from analysis of a subsequent measurement data of the individual at a later time point; and
determining a subsequent magnitude of a gap between the predicted health status and the subsequent health status of the individual.

8. The method according to claim 1, wherein the step of measuring further comprises determining a presence or absence of one or more polymorphisms in the Genomic Markers, wherein the one or more polymorphisms are associated with the disease or health risk.

9. The method according to claim 1, wherein the step of measuring further comprises comparing levels in the biological sample of the Proteomic Markers, the Metabolomic Markers, the Exposomic Markers or a combination thereof with levels of the corresponding markers from the published data from samples of the human subjects that have the disease or health risk, wherein the levels are correlated with having or at risk of developing the disease or health risk.

10. The method according to claim 1, wherein the confidence score is relates to a measure of confidence on the strength of predictiveness of the published data used to determine the likelihood of having or at risk of developing the disease or health risk.

11. The method according to claim 1, wherein the confidence score indicates a likelihood that the published data has reproducible results, and wherein the confidence score is weighted based on a comparison of a number of citations received by the published data and a number of references cited by the published data.

12. The method according to claim 1, wherein the Exposomic Markers are selected from the group consisting of: vitamin, amino acid, inorganic compound, biogenic amine, organic acid, amine oxide, hydrocarbon derivative and a combination thereof.

13. The method according to claim 12, wherein the vitamin is selected from the group consisting of: vitamin A, vitamin B3-amide, vitamin B6, vitamin B1, calcidiol, vitamin D2, vitamin B7, vitamin B5, vitamin B2 and a combination thereof.

14. The method according to claim 12, wherein the amino acid is selected from the group consisting of: branched chain amino acid, aromatic amino acid, aliphatic amino acid, polar side chain amino acid, acidic and basic amino acid, and unique amino acid preferably glycine and proline, and a combination thereof.

15. The method according to claim 12, wherein the inorganic compound is selected from the group consisting of: copper, iron, sodium, calcium, potassium, phosphorus, magnesium, strontium, rubidium, antimony, selenium, cesium, zinc, barium and a combination thereof.

16. The method according to claim 12, wherein the biogenic amine is selected from the group consisting of: trans-OH-proline, acetyl-ornithine, alpha-aminoadipic acid, beta-alanine, taurine, carnosine, methylhistidine and a combination thereof.

17. The method according to claim 12, wherein the organic acid is selected from the group consisting of: hippuric acid, 3-(3-hydroxyphenyl)-3-hydroxypropionic acid, 5-hydroxyindole-3-acetic acid, sarcosine, hydroxyphenylacetic acid and a combination thereof.

18. The method according to claim 12, wherein the amine oxide is trimethylamine N-oxide.

19. The method according to claim 12, wherein the hydrocarbon derivative is trigonelline.

20. The method according to claim 1, wherein the Metabolomic Markers are selected from the group consisting of: acylcarnitine, biogenic amine, lysophospholipid, glycerophospholipid, sphingolipid, organic acid, amino acid, sugar, hydrocarbon derivative and a combination thereof.

21. The method according to claim 20, wherein the Metabolic Markers are acylcarnitines selected from the group consisting of: long chain acylcarnitine, medium chain acylcarnitine, and short chain acylcarnitine and a combination thereof.

22. The method according to claim 20, wherein the Metabolic Marker are biogenic amines selected from the group consisting of: creatinine, kynurenine, methionine-sulfoxide, spermidine, spermine, asymmetric dimethylarginine, putrescine, serotonin and a combination thereof.

23. The method according to claim 20, wherein the Metabolic Markers are lysophosphatidylcholine.

24. The method according to claim 20, wherein the Metabolic Marker are glycerophospholipid.

25. The method according to claim 20, wherein the Metabolic Marker are sphingolipid selected from the group consisting of: sphingolipid, hydroxy fatty acid sphingomyelin and a combination thereof.

26. The method according to claim 20, wherein the Metabolic Markers are organic acids selected from the group consisting of: short chain fatty acid, medium chain fatty acid, long chain fatty acid and a combination thereof.

27. The method according to claim 20, wherein the Metabolic Markers are amino acids selected from the group consisting of: betaine, creatine, citric acid and a combination thereof.

28. The method according to claim 20, wherein the Metabolic Markers are glucose.

29. The method according to claim 20, wherein the Metabolic Markers are hydrocarbon derivatives selected from the group consisting of: lactic acid, pyruvic acid, succinic acid and a combination thereof.

30. The method according to claim 1, wherein the Proteomic Markers are selected from the group consisting of: blood clotting protein, cell adhesion protein, immune response protein, transport protein, enzyme, hormone-like protein and a combination thereof.

31. The method according to claim 30, wherein the blood clotting protein is selected from the group consisting of: Protein Z-dependent protease inhibitor, coagulation factor protein, Antithrombin-III, Plasma serine protease inhibitor, Plasminogen, Prothrombin, Carboxypeptidase B2, Kininogen-1, Vitamin K-dependent protein S, Alpha-2-antiplasmin, Fibrinogen gamma chain, Tetranectin, Heparin cofactor 2, Fibrinogen beta chain, Fibrinogen alpha chain, Vitamin K-dependent protein Z, Alpha-2-macroglobulin, Endothelial protein C receptor, von Willebrand Factor and a combination thereof.

32. The method according to claim 30, wherein the cell adhesion protein is selected from the group consisting of: Inter-alpha-trypsin inhibitor heavy chain H1, Cartilage acidic protein 1, Inter-alpha-trypsin inhibitor heavy chain H4, Proteoglycan 4, Fibronectin, Vitronectin, Attractin, Intercellular adhesion molecule 1, Lumican, Galectin-3-binding protein, Cadherin-5, Leucine-rich alpha-2-glycoprotein 1, Tenascin, Vasorin, Fibulin-1, Probable G-protein coupled receptor 116, L-selectin, Thrombospondin-1 and a combination thereof.

33. The method according to claim 30, wherein the immune response protein is selected from the group consisting of: Mannose-binding protein C, Complement component protein, Ficolin-2, Kallistatin, Plastin-2, Ig mu chain C region, Protein AMBP, CD44 antigen, Ficolin-3, IgGFc-binding protein, Mannan-binding lectin serine protease 2, Serum amyloid A-1 protein, Beta-2-microglobulin, Protein S100-A9, C-reactive protein and a combination thereof.

34. The method according to claim 30, wherein the transport protein is selected from the group consisting of: Apolipoprotein, Alpha-1-acid glycoprotein 1, Serum albumin, Retinol-binding protein 4, Hormone-binding globulin, Serotransferrin, Clusterin, Beta-2-glycoprotein 1, Phospholipid transfer protein, Beta-2-glycoprotein 1, Phospholipid transfer protein, Hemopexin, Inter-alpha-trypsin inhibitor heavy chain H2, Gelsolin, Transthyretin, Afamin, Histidine-rich glycoprotein, Serum amyloid A-4 protein, Lipopolysaccharide-binding protein, Haptoglobin, Ceruloplasmin, Vitamin D-binding protein, Hemoglobin subunit alpha 1 and a combination thereof.

35. The method according to claim 30, wherein the enzyme is selected from the group consisting of: Phosphatidylinositol-glycan-specific phospholipase D, Carboxypeptidase N subunit 2, Serum paraoxonase/arylesterase 1, Biotinidase, Glutathione peroxidase 3, Carboxypeptidase N catalytic chain, Cholinesterase, Xaa-Pro dipeptidase, Carbonic anhydrase 1, Lysozyme C, Peroxiredoxin-2, Beta-Ala-His dipeptidase and a combination thereof.

36. The method according to claim 30, wherein the hormone-like protein is selected from the group consisting of: Extracellular matrix protein 1, Alpha-2-HS-glycoprotein, Angiogenin, Insulin-like growth factor-binding protein complex acid labile subunit, Fetuin-B, Adipocyte plasma membrane-associated protein, Pigment epithelium-derived factor, Zinc-alpha-2-glycoprotein, Angiotensinogen, Insulin-like growth factor-binding protein 3, Insulin-like growth factor-binding protein 2 and a combination thereof.

37. The method according to claim 1, wherein the Genomic Markers are selected from the group consisting of Table 1 genes 1 to 477 or a combination thereof.

38. The method according to claim 8, wherein the one or more polymorphisms in the Genomic Markers are selected from the group consisting of Table 1 single nucleotide polymorphisms 1 to 477 or a combination thereof.

39. The method according to claim 2, wherein the health recommendations are output digitally to a computer display.

40. A method of determining a health status of an individual based on a set of Disease Risk Markers corresponding to a disease or health risk and a magnitude of a gap between measured Disease Risk Markers and published Disease Risk Markers, the method comprising:
analyzing at least 25, preferably at least 20, preferably at least 15, preferably at least 10 or preferably at least 5 sampled Disease Risk Markers of the individual to determine measurement data indicative of a disease or health risk or risk of developing thereof of an individual, wherein the at least 25, preferably at least 20, preferably at least 15, preferably at least 10 or preferably at least 5 measurement data corresponds to the disease or health risk;
determining the absence or presence of polymorphisms in the sampled Disease Risk Markers or levels of the sampled Disease Risk Markers from the measurement data from the individual; and
calculating, by a computer device, and based on the at least 25, preferably at least 20, preferably at least 15, preferably at least 10 or preferably at least 5 measurement data, a magnitude of a gap between the sample Disease Risk Markers and corresponding published Disease Risk Markers, wherein each Disease Risk Marker is correlated with affecting one or more of the disease or health risk, and
wherein the magnitude of the gap indicates the health status of the individual.

41. The method according to claim 40, wherein the disease or health risk or risk of developing thereof is determined based on applying a predictive equation, wherein the predictive equation corresponds to the disease or health risk or the risk of developing thereof and is determined by multivariate regression analysis of published data of human subjects that have the disease or health risk.

42. A method for assessing Body Functions of an individual, the method comprising:
providing a biological sample obtained from the individual;
measuring at least 25, preferably at least 20, preferably at least 15, preferably at least 10 or preferably at least 5 Disease Risk Markers in the biological sample selected from the group consisting of Genomic Markers, Proteomic Markers, Metabolomic Markers, Exposomic Markers and a combination thereof to provide measurement data from the sample in relation to the individual; and
determining a predicted health status corresponding to the Body Functions, by applying a predictive equation corresponding to the measurement data to the Body Functions,
wherein the predictive equation corresponds to the Body Functions and is determined by a computer implemented multivariate regression analysis of published data of human subjects that have the disease or health risk,
wherein the computer implemented multivariate regression analysis comprises calculating a confidence score of each of the published data of the human subjects and the published data comprises a plurality of measurements corresponding to each human subject to the Body Functions,
wherein the measurements are associated with biological pathways involving a network of Genomic Markers, Proteomic Markers, Metabolomic Markers, and/or Exposomic Markers and determined from published Disease Risk Markers of each human subject in the published data, and
wherein the predicted health status is representative of the Body Functions of the individual.

43. The method according to claim 42, wherein the step of determining Body Functions further comprises:
comparing the measured Disease Risk Markers to the published Disease Risk Markers associated with the Body Functions; and
determining a magnitude of a gap between the measured Disease Risk Markers and the published Disease Risk Markers.

44. A method of assessing the health status of an individual, the method comprising:
providing a biological sample obtained from the individual;
measuring at least 25, preferably at least 20, preferably at least 15, preferably at least 10 or preferably at least 5 Disease Risk Markers in the biological sample selected from the group consisting of Genomic Markers, Proteomic Markers, Metabolomic Markers, Exposomic Markers and a combination thereof to provide measurement data from the sample in relation to the individual; and
determining a predicted health status corresponding to a disease or health risk or a risk of developing thereof, by applying a predictive equation corresponding to the disease or health risk or the risk of developing thereof to the measurement data;
wherein the predictive equation is determined by a computer implemented multivariate regression analysis of published data of human subjects that have the disease or health risk,
wherein the computer implemented multivariate regression analysis comprises calculating a first confidence score of each of the published data of the human subjects, wherein the first confidence score relates to a measure of confidence on the strength of predictiveness of the published data used to determine the likelihood of having or at risk of developing the disease or health risk;
wherein the published data comprises a plurality of measurements corresponding to each human subject that have the disease or health risk,
wherein the measurements correspond to each Disease Risk Marker associated with the disease or health risk and determined from published Disease Risk Markers of each human subject in the published data, and
wherein the predicted health status is representative of the individual having the disease or health risk or the risk of developing thereof.

45. The method according to claim 44, further comprising
determining whether each of the Disease Risk Marker is conventionally used in diagnostic methods to determine the likelihood of having or at risk of developing the disease or health risk as determined by multivariate regression analysis of published data of the human subjects that have the disease or health risk, wherein the multivariate regression analysis comprises calculating an additional confidence score of the published data of the human subjects, wherein the additional confidence score relates to a measure of confidence of the use of each of the Disease Risk Marker in diagnostic methods to determine the likelihood of having or at risk of developing the disease or health risk; and
calculating a weighted confidence score of the published data based on inputs from all of the confidence scores.

46. The method according to claim 44, further comprising
determining whether each of the Disease Risk Marker is a component of an actionable pathway that can be targeted by a health recommendation as determined by multivariate regression analysis of the published data of the human subjects that have the disease or health risk, wherein the multivariate regression analysis comprises calculating an additional confidence score of the published data of the human subjects, wherein the additional confidence score relates to a measure of confidence that each of the Disease Risk Marker is the component of the actionable pathway that can be targeted by the health recommendation; and
calculating a weighted confidence score of the published data based on inputs from all of the confidence scores.

47. The method according to claim 44, wherein the predictive equation is further determined by multivariate regression analysis of controlled experiments of human subjects that have the disease or health risk and further comprising
determining whether a health recommendation for the disease or health risk can be validated in respect of efficacy as determined by multivariate regression analysis of the controlled experiments comprising exposing subjects to the health recommendation, wherein the determination comprises calculating an additional confidence score of each of the controlled experiment, wherein the additional confidence score relates to a measure of confidence that the health recommendation for the disease or health risk can be validated as effective; and
calculating a weight confidence score of the published data and the controlled experiments based on inputs from all of the confidence scores.

48. A system (100) for performing the method of any one of claims 1 to 47.

49. A system (100) for assessing the health status of an individual, the system comprising:
at least one processor (104);
an interface (106); and
at least one tangible, non-transitory computer readable storage medium storing computer executable instructions (108) that, when executed by the at least one processor (104), cause the system (100) to:
obtain, via a Disease Risk Markers measurement provider (115), an indication of the presence, absence or level of Disease Risk Markers in a biological sample from the individual, wherein the Disease Risk Marker is selected from the group consisting of Genomic Markers, Proteomic Markers, Metabolomic Markers, Exposomic Markers and a combination thereof; and
determine, based on the indication of the presence, absence or level of the sampled Disease Risk Markers, a predicated health status corresponding to a disease or health risk or a risk of developing thereof, by applying a predictive equation corresponding to the sampled Disease Risk Markers,
wherein the predictive equation is determined by multivariate regression analysis of published data of human subjects that have the disease or health risk,
wherein the multivariate regression analysis comprises calculating a first confidence score of each of the published data of the human subjects, wherein the first confidence score relates to a measure of confidence on the strength of predictiveness of the published data used to determine the likelihood of having or at risk of developing the disease or health risk, and the published data comprises a plurality of measurements corresponding to each individual that has the disease or health risk,
wherein the measurements are associated with the disease or health risk and determined from published Disease Risk Markers of each human subject in the published data, and
wherein the health status is representative of the individual having the disease or health risk or risk of developing thereof.

50. The system (100) according to claim 49, wherein the at least one tangible, non-transitory computer readable storage medium stores further comprises additional computer executable instructions (108) that, when executed by the at least one processor (104), cause the system (100) to make a health recommendation by:
identifying dietary changes, nutritional supplements, exercise actions or a combination thereof, suitable for improving the health status of the individual; and
presenting the identity of the dietary changes, the nutritional supplements, the exercise actions or the combination thereof at the interface (106).

51. The system (100) according to claim 49, wherein the at least one tangible, non-transitory computer readable storage medium stores further comprises computer executable instructions (108) that, when executed by the at least one processor (104), cause the system (100) to:
determine, based on the sampled Disease Risk Markers, a respective current health status corresponding to each disease or health risk included in the group of the diseases or health risk;
determine a respective magnitude of a respective gap between the respective predicted health status and the respective current health status for each disease or health risk included in the group of the diseases or health risk;
identify a specific disease or health risk associated with the determined gap magnitudes; and
identify dietary changes, nutritional supplements, exercise actions or a combination thereof, suitable for improving the specific disease or health risk.

52. The system (100) according to claim 49, wherein the at least one tangible, non-transitory computer readable storage medium stores further comprises computer executable instructions (108) that, when executed by the at least one processor (104), cause the system (100) to:
determine a subsequent health status of the individual from analysis of subsequent sampled Disease Risk Markers of the individual at a later time point; and
determine a subsequent magnitude of a gap between the predicted health status and the subsequent health status of the individual.

53. The system (100) according to claim 49, wherein the multivariate regression analysis further comprises calculating additional confidence scores on one or more measures selected from a measure of confidence of the use of each of the Disease Risk Marker's in diagnostic methods to determine the likelihood of having or at risk of developing the disease or health risk, or a measure of confidence that each of the Disease Risk Marker is a component of an actionable pathway that can be targeted by a health recommendation, and calculating a weighted confidence score of the published data based on inputs from all of the confidence scores.

54. The system (100) according to claim 53, wherein the predictive equation is further determined by multivariate regression analysis of controlled experiments of human subjects that have the disease or health risk, and wherein the multivariate regression analysis further comprises calculating an additional confidence score of each of the controlled experiment, wherein the additional confidence score relates to a measure of confidence that the health recommendation for the disease or health risk can be validated as effective, and calculating a weighted confidence score of the published data and the controlled experiments based on inputs from all of the confidence scores.

55. A system (120) comprising:
a) a database (121) comprising published data of Disease Risk Markets associated with a disease or health risk in human subjects, wherein the Disease Risk Markers are selected from group consisting of: Genomic Markers, Proteomic Markers, Metabolomic Markers, Exposomic Markers and a combination thereof;
b) a computer (122) comprising computer-readable instructions for determining a first confidence score of each of the published data, wherein the first confidence score indicates a likelihood of an association of the Disease Risk Markers to the disease or health risk in the published data is reproducible, wherein the computer-readable instructions:
(i) generate relational data to represent a relationship between each of the published Disease Risk Marker and the association; and
(ii) uses the relational data to determine the confidence score for the association.

56. The system (120) according to claim 55, wherein the relational data is based on a comparison of a number of citations received by the published data and a number of references cited by the published data.

57. The system (120) according to claim 55, wherein the computer-readable set of instructions further determine additional confidence scores of one or more measures of the published data and controlled experiments, wherein the one or more measures are selected from a measure of confidence of the use of each of the Disease Risk Marker's in diagnostic methods to determine the likelihood of having or risk of developing the disease or health risk, a measure of confidence that each of the Disease Risk Marker is a component of an actionable pathway that can be targeted by a health recommendation, or a measure of confidence that the health recommendation for the disease or health risk can be validated as effective, and calculates a weight confidence score of the published data and the controlled experiments based on inputs from all of the confidence scores.

58. A method for treating a disease or condition in a subject, the method comprising: determining a health status of an individual according to the method of any one of claims 1 to 47, wherein said health status is indicative of the progression of the disease or condition, and recommending changes in medication, supplements and/or nutrition for the individual to treat the disease or condition.

59. The method according to claim 58, wherein the disease or condition is selected from the group consisting of psoriasis, crohn's disease, bipolar disorder, depression, schizophrenia, age-related macular degeneration, adolescent idiopathic scoliosis, hurler syndrome, tooth agenesis, celiac disease, multiple sclerosis, vas deferens condition, asthma, allergic rhinitis, heroin addition, low bone mineral density, osteoporosis, gout, ADHD, ulcerative colitis, pancolitis, post-traumatic stress disorder, autism, type 1 diabetes, type 2 diabetes, renal cell carcinoma, peanut allergy, Fuch's dystrophy, Creutzfeldt-Jakob disease, hepatitis C, obsessive-compulsive disorder, coronary artery disease, cardiovascular disease, pancreatic cancer, systemic lupus erythematosus, rheumatoid arthritis, cocaine dependence, deep vein thrombosis, Hirschsprung disease, nicotine dependence, diabetic nephropathy, ischemic stroke, T2D, autoimmune disease, several alcohol withdrawal, Atrial Fibrillation, ankylosing spondylitis, melanoma, ALS, migraine-associated vertigo, endometrial ovarian cancer, coronary heart disease, Parkinson's Disease, lung cancer, prostate cancer, childhood-onset steroid-sensitive nephrotic syndrome, schizophrenia, phobic disorders, Graves' disease, obesity, wet ARMD, docetaxel-induced nephropathy, pulmonary tuberculosis, male pattern baldness, bipolar disorder, CRP, osteoarthritis, Parkinson's Disease, serum uric acid concentration, myocardial infarction risk, intracranial aneurysm risk, metabolic syndrome, spondylitis, hyper triglyceride, lupus, ischemic stroke, otosclerosis, cutaneous melanoma, ADHA, non-alcoholic fatty liver disease, atherosclerotic cerebral infarction, restless legs syndrome, narcolepsy, temporomandibular joint disorder (TMD), colorectal cancer, Ankylosing Spondylitis, neuroticism, panic disorder, venous thrombosis, glaucoma, hereditary hemochromatosis, Bechet's disease, hypertension, insulin sensitivity, anorexia, Tourette's syndrome, primary biliary cirrhosis, intracranial aneurysm, vitiligo, alcohol dependence, glioma, high blood pressure, hyperuricemia, pulmonary tuberculosis, spondylitis, venous thromboembolism, lumbar disc disease, cardiomyopathy, primary sclerosing cholangitis, colorectal caner, esophageal cancer and breast cancer.

60. Any of the claims 1 to 59 in combination with any number of any other of the claims 1 to 59.
